SEQUENCE LISTING (92 sequences)

SEQ ID NO: 1 - protein, 69 aa, Actinoplanes teichomyceticus
MTNPFDNEDGSFLVLVNGEGQHSLWPAFAEVPDGWTGVHGPASRQDCLGYVEQNWTDLRPKSLISQISD

SEQ ID NO: 2 - protein, 69 aa, Actinoplanes teichomyceticus
MTNPFDNEDGSFLVLVNGEGQHSLWPAFAEVPDGWTGVHGPASRQDCLGYVEQNWTDLRPRSLVEQADA

SEQ ID NO: 3 - protein, 69 aa, Unknown
  Uncultured soil bacterium
MTNPFDNEDGTFFVLVNDEGQHSLWPTFAEVPAGWTRVHGEATRQECLAYVEENWTDLRPKSLIQALGA

SEQ ID NO: 4 - protein, 69 aa, Unknown
  Uncultured soil bacterium
MTNPFDNEDGSFFVLVNDEGQHSLWPTFAEVPAGWVCVYGEATRQECLTFVEENWTDLRPKSLIQEVGG

SEQ ID NO: 5 - protein, 69 aa, Amycolatopsis balhimycina
MSNPFDNEDGSFFVLVNDEGQHSLWPTFAEVPAGWTRVHGEAGRQECLAYVEENWTDLRPKSLIREASA

SEQ ID NO: 6 - protein, 73 aa, Streptomyces lavendulae
MTNPFDNENGTFLVLVNDEGQHSLWPVFAEIPQGWTTAFGEASRAECLEFVEQNWTDMRPKSLVARMEGTATA

SEQ ID NO: 7 - protein, 70 aa, Streptomyces coelicolor
MSTNPFDDADGRFLVLVNDEGQHSLWPAFAAVPGGWTTVFEENTRDACLAYVEANWTDLRPRSLARTADA

SEQ ID NO: 8 - DNA, 955 bp, Artificial Sequence
  “Synthetic”
  Synthetic
gcggccgcaaagcttgatatcgaattccttatactgggcctgctgcattggtctgccattgcagggtatatatggctgacctggccaatctccatcgagaatctgggcgactgaagcactgcccgcagacaagatggagactttcgtctagcacggtctagggcagatccgatgccattggctctgtcaactgtcgactacatgtatctgcatgttgcatcgggaaatcccaccacagggacagccaagcggccccgcgacttggcagtgggcaaactacgcccgattctggtgccaagaaccgagaagaatgagacagacccacgttgcactctaaccggatgctatcgacttacggtggctgaagattcaacacgctgcaacgagagccaaggtggtccggacattttctacgtgccggtttaccttggaacatcgccgtcgttgagtgcacgttgccttctctctcgtggcttggctgggcccacgagcccgattgactcgacggcgttacttgggtatctatggccccgttttctggcacggtaatgataagtacttactagtcttcgagcggggaagtgttgctctgcccgagcatcaacgattggcctgatcgcaccgtctgcaaatgccacggtgcggaccgactgaaatctcagaccaccaaagaccctccgacttcgagatacggttactaattttacactggctccagcggccccatccagtaagcatctgggctgcaagcgtataatgtctccaggttgtctcagcataaacaccccgcccccgctcaggcacacaggaagagagctcaggtcgtttccattgcgtccatactcttcactcattgtcatcgcaggagaacttcccctgtccctttgccaagccctctcttcgtcgttgtccacgccttcaagttttcaccattatttttctagacacccatatg

SEQ ID NO: 9 - DNA, 538 bp, Artificial Sequence
  “Synthetic”
  Synthetic
atgcatcttttgtatgtagcttcaaccgactccgtcttcacttcttcgcccgcactgcctaccgtttgtaccatctgactcatataaatgtctagcccctacctacactatacctaagggagagaagcgtagagtgattaacgtacgggcctatagtaccccgatctctagatagaacatttagtagagattaggatgcctaactaatttaacttgagcattgtcccgttcatattgattttcagtccattatacactcttaatcgtttcccggtagaagcctgatatatacgaccatagggtgtggagaacagggcttcccgtctgcttggccgtacttaagctatatattctacacggccaatactcaatgtgcccttagcacctaagcggcactctagggtaagtgcgggtgatataggtgagaagtcttaagactgaagacaggatatcacgcgttaccctgcaccgtacctactaccttcaatatcaactctttcaggatggacagggtcgacactagttctagagcggccgc

SEQ ID NO: 10 - DNA, 219 bp, Artificial Sequence
  “Synthetic”
  Synthetic
catatgacgaacccatttgacaacgaggacggatcatttttggtattggttaacggtgaaggtcaacacagcctgtggccggcattcgccgaggtgccggatggctggaccggcgttcatggtccggcgagccgccaagactgcctgggttacgtcgaacagaattggaccgatctgcgtccgaaaagcctgatctcccagattagcgattgaatgcat

SEQ ID NO: 11 - DNA, 219 bp, Artificial Sequence
  “Synthetic”
  Synthetic
catatgaccaacccgttcgacaacgaggacggcaccttcttcgtgctggtcaacgacgagggccagcactccctctggccgaccttcgccgaggtgcctgccggctggacccgcgtgcacggtgaagccacccggcaggagtgcctcgcgtatgtcgaggagaactggacggacctgcggccgaagagcctcatccaggccctcggcgcctgaatgcat

SEQ ID NO: 12 - DNA, 231 bp, Artificial Sequence
  “Synthetic”
  Synthetic
catatgactaacccgttcgacaacgagaacggcactttccttgttcttgttaacgacgagggacagcactctctttggccggttttcgctgagattccgcagggctggactactgctttcggcgaggcttctcgtgctgagtgccttgagttcgttgagcagaactggactgacatgcgtccgaagtctcttgttgctcgtatggagggcactgctactgcttgaatgcat

SEQ ID NO: 13 - DNA, 219 bp, Artificial Sequence
  “Synthetic”
  Synthetic
catatgactaacccgttcgacaacgaggacggctctttcttcgttcttgttaacgacgagggccagcactctctttggccgactttcgctgaggttccggctggctgggtttgcgtttacggcgaggctactcgtcaggagtgccttactttcgttgaggagaactggactgaccttcgtccgaagtctcttattcaggaggttggcggctgaatgcat

SEQ ID NO: 14 - DNA, 222 bp, Artificial Sequence
  “Synthetic”
  Synthetic
catatgtctactaacccgttcgacgacgctgacggccgtttccttgttcttgttaacgacgagggccagcactctctttggccggctttcgctgctgttccgggcggctggactactgttttcgaggagaacactcgtgacgcttgccttgcttacgttgaggctaactggactgaccttcgtccgcgttctcttgctcgtactgctgacgcttgaatgcat

SEQ ID NO: 15 - DNA, 26 bp, Artificial Sequence
  “Synthetic”
  Synthetic
gtctgccattgcagggtatatatggc

SEQ ID NO: 16 - DNA, 30 bp, Artificial Sequence
  “Synthetic”
  Synthetic
cgggacaatgctcaagttaaattagttagg

SEQ ID NO: 17 - protein, 44 aa, Artificial Sequence
  Synthetic
  Synthetic
  SITE 2,4,6,8,11,12,13,14,15,17,20,21,22,23,24,25,26,27,28,29,30,31,33,34,35,36,37,38,42: Xaa is unknown
NXEXQXSXWPXXXXXPXGWXXXXXXXXXXXXLXXXXXXWTDXRP

SEQ ID NO: 18 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAEVPDGWXXXXXXXXXXXXLXXXXXXWTDXRP

SEQ ID NO: 19 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAEVPAGWXXXXXXXXXXXXLXXXXXXWTDXRP

SEQ ID NO: 20 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAEVPGGWXXXXXXXXXXXXLXXXXXXWTDXRP

SEQ ID NO: 21 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAEVPQGWXXXXXXXXXXXXLXXXXXXWTDXRP

SEQ ID NO: 22 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAAVPDGWXXXXXXXXXXXXLXXXXXXWTDXRP

SEQ ID NO: 23 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAAVPAGWXXXXXXXXXXXXLXXXXXXWTDXRP

SEQ ID NO: 24 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAAVPGGWXXXXXXXXXXXXLXXXXXXWTDXRP

SEQ ID NO: 25 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAAVPQGWXXXXXXXXXXXXLXXXXXXWTDXRP

SEQ ID NO: 26 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAEIPDGWXXXXXXXXXXXXLXXXXXXWTDXRP

SEQ ID NO: 27 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAEIPAGWXXXXXXXXXXXXLXXXXXXWTDXRP

SEQ ID NO: 28 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAEIPGGWXXXXXXXXXXXXLXXXXXXWTDXRP

SEQ ID NO: 29 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPAFAEIPQGWXXXXXXXXXXXXLXXXXXXWTDXRP

SEQ ID NO: 30 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAEVPDGWXXXXXXXXXXXXLXXXXXXWTDXRP

SEQ ID NO: 31 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAEVPAGWXXXXXXXXXXXXLXXXXXXWTDXRP

SEQ ID NO: 32 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAEVPGGWXXXXXXXXXXXXLXXXXXXWTDXRP

SEQ ID NO: 33 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAEVPQGWXXXXXXXXXXXXLXXXXXXWTDXRP

SEQ ID NO: 34 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAAVPDGWXXXXXXXXXXXXLXXXXXXWTDXRP

SEQ ID NO: 35 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAAVPAGWXXXXXXXXXXXXLXXXXXXWTDXRP

SEQ ID NO: 36 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAAVPGGWXXXXXXXXXXXXLXXXXXXWTDXRP

SEQ ID NO: 37 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAAVPQGWXXXXXXXXXXXXLXXXXXXWTDXRP

SEQ ID NO: 38 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAEIPDGWXXXXXXXXXXXXLXXXXXXWTDXRP

SEQ ID NO: 39 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAEIPAGWXXXXXXXXXXXXLXXXXXXWTDXRP

SEQ ID NO: 40 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAEIPGGWXXXXXXXXXXXXLXXXXXXWTDXRP

SEQ ID NO: 41 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPTFAEIPQGWXXXXXXXXXXXXLXXXXXXWTDXRP

SEQ ID NO: 42 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAEVPDGWXXXXXXXXXXXXLXXXXXXWTDXRP

SEQ ID NO: 43 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAEVPAGWXXXXXXXXXXXXLXXXXXXWTDXRP

SEQ ID NO: 44 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAEVPGGWXXXXXXXXXXXXLXXXXXXWTDXRP

SEQ ID NO: 45 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAEVPQGWXXXXXXXXXXXXLXXXXXXWTDXRP

SEQ ID NO: 46 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAAVPDGWXXXXXXXXXXXXLXXXXXXWTDXRP

SEQ ID NO: 47 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAAVPAGWXXXXXXXXXXXXLXXXXXXWTDXRP

SEQ ID NO: 48 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAAVPGGWXXXXXXXXXXXXLXXXXXXWTDXRP

SEQ ID NO: 49 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAAVPQGWXXXXXXXXXXXXLXXXXXXWTDXRP

SEQ ID NO: 50 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAEIPDGWXXXXXXXXXXXXLXXXXXXWTDXRP

SEQ ID NO: 51 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAEIPAGWXXXXXXXXXXXXLXXXXXXWTDXRP

SEQ ID NO: 52 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAEIPGGWXXXXXXXXXXXXLXXXXXXWTDXRP

SEQ ID NO: 53 - protein, 44 aa, Artificial Sequence
  Synthetic
  Synthetic
  SITE 2,4,6,8,20,21,22,23,24,25,26,27,28,29,30,31,33,34,35,36,37,38,42: Xaa is unknown
NXEXQXSXWPVFAEIPQGWXXXXXXXXXXXXLXXXXXXWTDXRP

SEQ ID NO: 54 - protein, 3791 aa, Penicillium chrysogenum
MTQLKPPNGTTPIGFSATTSLNASGSSSVKNGTIKPSNGIFKPSTRDTMDPCSGNAADGSIRVRFRGGIERWKECVNQVPERCDLSGLTTDSTRYQLASTGFGDASAAYQERLMTVPVDVHAALQELCLERRVSVGSVINFSVHQMLKGFGNGTHTITASLHREQNLQNSSPSWVVSPTIVTHENRDGWSVAQAVESIEAGRGSEKESVTAIDSGSSLVKMGLFDLLVSFVDADDARIPCFDFPLAVIVRECDANLSLTLRFSDCLFNEETICNFTDALNILLAEAVIGRVTPVADIELLSAEQKQQLEEWNNTDGEYPSSKRLHHLIEEVVERHEDKIAVVCDERELTYGELNAQGNSLARYLRSIGILPEQLVALFLDKSEKLIVTILGVWKSGAAYVPIDPTYPDERVRFVLDDTKARAIIASNQHVERLQREVIGDRNLCIIRLEPLLASLAQDSSKFPAHNLDDLPLTSQQLAYVTYTSGTTGFPKGIFKQHTNVVNSITDLSARYGVAGQHHEAILLFSACVFEPFVRQTLMALVNGHLLAVINDVEKYDADTLLPFIRRHSITYLNGTASVLQEYDFSDCPSLNRIILVGENLTEARYLALRQRFKNRILNEYGFTESAFVTALKIFDPESTRKDTSLGRPVRNVKCYILNPSLKRVPIGATGELHIGGLGISKGYLNRPELTPHRFIPNPFQTDCEKQLGINSLMYKTGDLARWLPNGEVEYLGRADFQIKLRGIRIEPGEIETMLAMYPRVRTSLVVSKKLRNGPEETTNEHLVGYYVCDSASVSEADLLSFLEKKLPRYMIPTRLVQLSQIPVNVNGKADLRALPAVDISNSTEVRSDLRGDTEIALGEIWADVLGARQRSVSRNDNFFRLGGHSITCIQLIARIRQRQRLSVSISVEDVFATRTLERMADLLQNKQQEKCDKPHEAPTELLEENAATDNIYLANSLQQGFVYHYLKSMEQSDAYVMQSVLRYNTTLSPDLFQRAWKHAQQSFPALRLRFSWEKEVFQLLDQDPPLDWRFLYFTDVAAGAVEDRKLEDLRRQDLTERFKLDVGRLFRVYLIKHSENRFTCLFSCHHAILDGWSLPLLFEKVHETYLQLLHGDNLTSSMDDPYTRTQRYLHAHREDHLDFWAGVVQKINERCDMNALLNERSRYKVQLADYDQVQEQRQLTIALSGDAWLADLRQTCSAQGITLHSILQFVWHAVLHAYGGGTHTITGTTISGRNLPILGIERAVGPYINTLPLVLDHSTFKDKTIMEAIEDVQAKVNVMNSRGNVELGRLHKTDLKHGLFDSLFVLENYPNLDKSRTLEHQTELGYSIEGGTEKLNYPLAVIAREVETTGGFTVSICYASELFEEVMISELLHMVQDTLMQVARGLNEPVGSLEYLSSIQLEQLAAWNATEAEFPDTTLHEMFENEASQKPDKIAVVYEETSLTYRELNERANRMAHQLRSDVSPNPNEVIALVMDKSEHMIVNILAVWKSGGAYVPIDPGYPNDRIQYILEDTQALAVIADSCYLPRIKGMAASGTLLYPSVLPANPDSKWSVSNPSPLSRSTDLAYIIYTSGTTGRPKGVTVEHHGVVNLQVSLSKVFGLRDTDDEVILSFSNYVFDHFVEQMTDAILNGQTLLVLNDGMRGDKERLYRYIEKNRVTYLSGTPSVVSMYEFSRFKDHLRRVDCVGEAFSEPVFDKIRETFHGLVINGYGPTEVSITTHKRLYPFPERRMDKSIGQQVHNSTSYVLNEDMKRTPIGAVGELYLGGEGVVRGYHNRADVTAERFIPNPFQSEEDKREGRNSRLYKTGDLVRWIPGSSGEVEYLGRNDFQVKIRGLRIELGEIEAILSSYHGIKQSVVIAKDCREGAQKFLVGYYVADAALPSAAIRRFMQSRLPGYMVPSRLILVSKFPVTPSGKLDTKALPPAEEESEIDVVPPRSEIERSLCDIWAELLEMHPEEIGIYSDFFSLGGDSLKSTKLSFMIHESFNRAVSVSALFCHRTVEAQTHLILNDAADVHEITPIDCNDTQMIPVSRAQERLLFIHEFENGSNAYNIDAAFELPGSVDASLLEQALRGNLARHEALRTLLVKDHATGIYLQKVLSPDEAQGMFSVNVDTAKQVERLDQEIASLSQHVFRLDDELPWEARILKLESGGLYLILAFHHTCFDAWSLKVFEQELRALYAALQKTKSAANLPALKAQYKEYALYHRRQLSGDRMRNLSDFWLRKLIGLEPLQLITDRPRPVQFKYDGDDLSIELSKKETENLRGVAKRCKSSLYVVLVSVYCVMLASYANQSDVSVGIPVSHRTHPQFQSVIGFFVNLVVLRVDISQSAICGLIRRVMKELVDAQLHQDMPFQEVTKLLQVDNDPSRHPLVQNVFNFESRANGEHDARSEDEGSLAFNQYRPVQPVDSVAKFDLNATVTELESGLRVNFNYATSLFNKSTIQGFLHTYEYLLRQLSELSAEGINEDTQLSLVRPTENGDLHLPLAQSPLATTAEEQKVASLNQAFEREAFLAAEKIAVVQGDRALSYADLNGQANQLARYIQSVSCIGADDGIALMLEKSIDTIICILAIWKAGAAYVPLDPTYPPGRVQLILEEIKAKAVLVHSSHASKCERHGAKVIAVDSPAIETAVSQQSAADLPTIASLGNLAYIIFTSGTSGKPKGVLVEQKAVLLLRDALRERYFGRDCTKHHGVLFLSNYVFDFSVEQLVLSVLSGHKLIVPPAEFVADDEFYRMASTHGLSYLSGTPSLLQKIDLARLDHLQVVTAAGEELHATQYEKMRRRFNGPIYNAYGVTETTVYNIIAEFTTNSIFENALREVLPGTRAYVLNAALQPVPFDAVGELYLAGDSVTRGYLNQPLLTDQRFIPNPFCKEEDIAMGRFARLYKTGDLVRSRFNRQQQPQLEYLGRGDLQIKMRGYRIEISEVQNVLTSSPGVREGAVVAKYENNDTYSRTAHSLVGYYTTDNETVSEADILTFMKARLPTYMVPSHLCCLEGALPVTINGKLDVRRLPEIINDSAQSSYSPPRNIIEAKMCRLWESALGMERCGIDDDLFKLGGDSITSLHLVAQIHNQVGCKITVRDIFEHRTARALHDHVFMKDSDRSNVTQFRTEQGPVIGEAPLLPIQDWFLSKALQHPMYWNHTFYVRTPELDVDSLSAAVRDLQQYHDVFRMRLKREEVGFVQSFAEDFSPAQLRVLNVKDVDGSAAVNEILDGWQSGFNLENGPIGSIGYLHGYEDRSARVWFSVHHMAIDTVSWQILVRDLQTLYRNGSLGSKGSSFRQWAEAIQNYKASDSERNHWNKLVMETASSISALPTSTGSRVRLSRSLSPEKTASLIQGGIDRQDVSVYDSLLTSVGLALQHIAPTGPSMVTIEGHGREEVDQTLDVSRTMGWFTTMYPFEIPRLSTENIVQGVVAVSERFRQVPARGVGYGTLYGYTQHPLPQVTVNYLGQLARKQSKPKEWVLAVGDNEFEYGLMTSPEDKDRSSSAVDVTAVCIDGTMIIDVDSAWSLEESEQFISSIEEGLNKILDGRASQQTSRFPDVPQPAETYTPYFEYLEPPRQGPTLFLLPPGEGGAESYFNNIVKRLRQTNMVVFNNYYLHSKRLRTFEELAEMYLDQVRGIQPHGPYHFIGWSFGGILAMEMSRRLVASDEKIGFLGIIDTYFNVRGATRTIGLGDTEILDPIHHIYNPDPANFQRLPSATDRIVLFKAMRPNNKYESENQRRLYEYYDALDSTDWTACYQAIPTSSWSRLRTIHTFPGSEIHNRWSRCVRLSRNTSLAIDPSLAAQYIGRWK

SEQ ID NO: 55 - protein, 2382 aa, Penicillium chrysogenum
MAAPSISPLFAPQMGVQRDFQDSEMQIATVNFPCTGQSNTSLANWLQQEEKSHLQLLQAAWSISLRSYTGSNDVLFSCLNTKDTAALGANSVVYVDEGNEDKYGSARRGNLREFDLAICFSKQNEANCPVIGIHHKPSVISTDFATMIAATVAKAIEEIVLHVDSLIASLDICSDADINCISRWNSPSDDGIPSAQCIHHIISQKCATQPESIAVSAWDGRLTYAELDGLSSSLAIRLQHLGVCQEIFVPLIFDKSKWAVIALLSVLKAGGAYFFLNPSNPIQYNLGLCSSLSPEVALCSPRHSTLAKSFAGTAIPVGEEHCELPESLPVDEKTPPCTAETTPSNAMYITFTSGTTGVPKGITTEHSAFYSMAMANGKALQVGPATRMLQFASYTFDVSNRDMLITLMFGGCICIPSELDRLNDLSGFINRQSVNLASLTPSLASTLNPALCPSLQGLVLGGESMNDSHISAWANHVRLFNAYGVSESAGIAALASDIQADYSPGNIGFGSGSTLWVVTIDQPDKLAPIGALGEMVIEGPSVARGYLGDKKRTEEQFTSTSKWKNRIRAQLSESRSSKRAFHTGDLVRYNLDGSLNFLGRKDHQVKIHGQRLELTAIEHHIAACLEVAESGFLHVAVVTAKNEGNGSVKLLAFLGLYTSRGSDSPSQLVSKKLEDVEALKVALRQHLLLCLPAFMVPVDFIFVQHMPLTTSGKINRLLLQEAAGHALLDDQKRNIDTSDLNGNQTPTTQNQRILIQSWAKALGIKSESIMRNDCFFRRGGDSIAAIKMAASLRQQELIISVSDVFKFSTFSDMASVLVKDHRPLQTAMLAPFSLIDNSQTVLDAVMEELGTGIDQIEDVYPCTHMQQGLIALTAQQPHSYIGRYTWQLAEMLDVEKFKNAWESAWLHNPILRTRIVQIPDGVFQVVVKTDMPWNTVTDISRGGDGKELREIDISNGPLIQFYLSKESFRLDIHHSLFDEWSLGLIMGQVERAYAGGGLRMQPFSPFVQHLLHERDTSLEDFWRQEFSGLQAEHFPAIASRPLSVEHPTEKVVLEHSVQLETGFSTKYTISSIVRLAWAIVLWHQTGSEDVVFGATVSGRNANIDGIDQLSGPTLATLPVRIKLAASQPIHEGLSQVQGQFVNMMVHEQAGLPRIRQVGREAAEACNFQNLLVVQPYEEQTESHMFKASANSASSSENAKSFASYPMVLICRPEKSGISMKAAFDPAILTPAAGHSILKQMSHVIQQLVTSDSTCIAAVSLVPPEDMATLRQWNHSLPNGVNTCIHARIQELCIGQPDTLAIHSQNLDLTYGQLDNYSDQFAQNLIGSGVKQGDFVPLFLERSPWVPVIMLAVLKTGAAFVLLDLSHPMQRLRTMCSMIDARIVVTSKEHADRSGNLLLPVIIFDPEAHAQNVSKQATAPELKPLTAVTTPDAPACVVFSSGSTGLPKGIVLPHSALTTSAAVMREYGMLGPKSRVFHFASFAFDISIGEILFTLAAGACVCVPHEEERKGNPAKAAGDLKVTWALLTPSVINLFDPSDVPTLEVLGSAGEPLTPQIVDTWAHRVKLYGMYAPAECTVISHIGRILPDTHHSNIGKSHGGVSWVVDPSDHNRLVPIGTVGELIVEGPTVSSGYLNDPAKTNEVFITSPSWLDEVRSHSGKMYKTGDLVRQTSEGSLEFVGRKDDQVKLHGQRLEVGEVEHCITSSCTAIKTATVECIKIREQNSRVSLVAFICPQTDEDWGQSLNDPSSEVGDLELISPPRDQFYSMIESLETSLRELLPAYMVPSFFVPLADVPLSLSGKVNRRLLRDQSTSWPMKRLGLYQLRRKSIPAEEVPVSIHGRKVQEIVGQALNLDPKSIPMNSNFFGLGGDSISAMQVSMLARRRGIRLTVADIFTQQTLSGLSLKCATENGDASQASKSRSLGRELPGSNIKSLHRCEIPRDKLPRQLPQEIADNIVEAMPATEFQTMTLHNFYSRYLWISLPERVNQEHLLNACDQLVQKHSVLRTVFYTNDDKSVVQLTLRKVPVNFVHYSNIENLEKHCADDSLAMGVPINSVPGFEVQLVTLRDSGMYLILRLPHAQFDGVSLDIICSDLSAAYSGDSLPPCAQFSDHIRHVWEKRIPESYNAWREVLGNVPMTSLNNKYLRNWGSASEMGSPNMGTDPDQPKVVTAMAETLPISPPPNITLATLVKLAWAITLSRLFTSIEEDDGASDDVVFGQVVHGRGLGISHEDRIVGPCLNIIPVRVHLPPRSNKLDLLGQVQQQHIQTMSVENLELGEITRNCTSWKAGTKFGSFIRFQNFTNNDDSTCSFDGSACETGLYSLPNRPSNTANVLVVPHGPTLSITMTISNQVLDRGSADFVAGYFSDVIESLASEETVCEYLE

SEQ ID NO: 56 - protein, 2372 aa, Penicillium chrysogenum
MDSYLGRDTISFPLSKELEAEWTDNGEVLLRSHLLKACWALLLGRLSETHTITFAVLEQLVAPPRDACSKLVASSPHLETWQISDRPDSLLVDAAKETHREPLSGTQTSTAVVIRWHDELNGPTSLPYAFNAIVVLDCSISPAKVYMEYSRASLTVNQAWSQLTATVHILEQLVMSPGISLRELDFLGSYSTKLILQWNNPYSLARPQVCIHTLILEHCRSQPDAEALCAWDGSVTYAELDRFSLAVAHQLLSLGVGPESVVPLYFEKSRWTVVAMLGVLRAGGAFVLLDPSHPMPRLAEICSEVQATVVITSESLQELGRKLGPRAVTILDTINSHVDTGRNAFNTSVKPSNAAYVAFTSGSTGKPKGIVIEHQCFVANTLAQNAVQNINSQTRAFQFASYGFDSSILETLMTLVAGGCVCIPSEKQRLNGLADAIRGMRANWLELTPSVARFINPEEVPDVSSVLLVGEPMSQDHITQWSGSGKIQLLNAYGPAECSVVATVQPNVQLEDPQNIGCSYSSHCWITNPQDHDQLEPLGAVGELLISGPIVARGYLNQPHQKSFISNPRWATRFGIPPGERVYKTGDLVRYNLDDGALRYVGRKDREVKIHGQRVDLHEIEHHASRFQKGMLAVADVLQVNGGSAGKLLALFIVADNDETRMTKESFVVPMNDTLLDLVTSIKHWLRDCLPPYMIPTKYTFVNRFPLTRTGKLDRRALVDLGAASSHSSTREQPSNQRDKEDVELDPGLSAKENTLCSVFAEALGCLAINIGPEDGFYDMGGNSLAAIELVARARNHGLEITVADVIRLQNPRKIARCTAESKDVREISPFSLLVDTEQSLSAATAQCGIGREMIEDIYPCTPLQEGLMHLSITNPGAFMGTYRFSLAPSTDLYRVWAAWEQLWLVHPILRTRIIQLQDGQKLQVVTKQKLPFEDISGMDNCQPMNLGTPLARVTYHRGRGSSGSDSGIFLLTMHHALFDGWSYLQMLGDLQVIYAGDKLSPRPSFKHAINYISKLSIEEGRSFWSQELKDFQATMFPTSSRRPTTSPHWQVRSQQIILAESDMNWTLANKIKLAWTLVISSQTHSNDVVYGLTVSGRNAPVPEIDRIVGPTFATFPFRTQLEDDISVEDMLVQMRQHDVSIMPFEHVGLRRIAESSSDAALACGFQNLLTIRLQSLQMPPGALIELPENENHDLKFASYALSIVAQQEGTSLGVKAIFNSCILGADRTEALLEQFDTLLQRILREPGTKMKDLRTQLSPEWQQLAAINKKSPSHLRCLHDIINHFSITQPNSEAVCAWDGSLTYSELVALARRLAGLLQSFGSGSEPGAVIGICVERSKWFPVAILGVMMSGAAMVLLEPNFPTQRLRHILRDAGARTMICSTVFQEKCAGLVDDMLVLTHDIVTQADYDAWTPSAVSHHDPMYVAFTSGSTGAPKGVVIEHGMVYSMLKAHKDIIGASIASRGLLFASPAFDICLAEIVLILCSGGCVCVPSEAQRMNSLAKTMTTMQVNMAMLTPSVARTLAPAAIPCLQTLILGGEAPSASDLETWASRVKLHQSYGPAECAMYTTTTHPLTSSSDLGNVGSSQNASCWIVDPDNHDELQPVGSIGELLIGGPIVGRGYINRAQESAAAFICDPVWSENFPFLQGARLYKTGDLAILNADGSLNLVGRKDTQVKLNGQRIELHEIEHCAERYQHGTAVIAELIKPVGIQRPRLIMFVYDPATVETTVGIDSTCHDHRGLFLPPSRQNQAYLEGVRDHLNQHLPPYMIPSHFLSLSRLPLSPSGKADRKTLRQVASKMDRETLEMYLDNPVAEKRKPTTEQERFVRASFATALSLNEEAIGMDDSFFALGGDSITAMRVLTLCRRRNMAISMQEFLSKNTVTLFCKHVILIQGQAVDSKRQKLLDSQDLVRGEDHLVQFQHLDYQLDMVRSQLNLLKSDSIQEIYPCSDAHSGVLELYTSNYTSTAIFEIRATGSVTPMQVSNAWSQLVHRHVALRTVLMKEPKVHADYLHVVLDKGPAQILALPRSKNALSELKGLEPVKSWGLSPPHRLIIGHDHSGTLFMRLETGYALIDAFSMTILLEELSLLLQGQPLPERGVSYREYLSNLRSQSSAETLQYWTQVLYGVYPSHLPRVPVTQSPLPEPRSQSRCLPFAQSKRLDSFWRSNNLTITNVFQLAWALTLAHYTNSRDVCFGTITSGRDIPHLEVWNIVGSFFNVLPCRIAIEPTRTVIDTLRQNQEDIQRRNDHQYCSIPDMIRKSGIRSLDNNQQLFNTVLTVQNPFSIQSSTAKDGSNEIDVKLIDLEDATEYDLCVAILPSPSHLKVELRYWSTTVSEGYASDILDRLFSQLEQIVHHATKPDFVQVYECTKH

SEQ ID NO: 57 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAEVPDGWXXXXXXXXXXXXLXXXXXXWTDXRP

SEQ ID NO: 58 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAEVPAGWXXXXXXXXXXXXLXXXXXXWTDXRP

SEQ ID NO: 59 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAEVPGGWXXXXXXXXXXXXLXXXXXXWTDXRP

SEQ ID NO: 60 - protein, 44 aa, Artificial Sequence
  “Synthetic”
  Synthetic
  SIGNAL 2,4,6,20,21,22,23,24,25,26,27,28,29,30,31,33,34,35,36,37,38,42: Xaa is unknown
NXEXQXSLWPAFAEVPQGWXXXXXXXXXXXXLXXXXXXWTDXRP

SEQ ID NO: 61 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAAVPDGWXXXXXXXXXXXXLXXXXXXWTDXRP

SEQ ID NO: 62 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAAVPAGWXXXXXXXXXXXXLXXXXXXWTDXRP

SEQ ID NO: 63 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAAVPGGWXXXXXXXXXXXXLXXXXXXWTDXRP

SEQ ID NO: 64 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAAVPQGWXXXXXXXXXXXXLXXXXXXWTDXRP

SEQ ID NO: 65 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAEIPDGWXXXXXXXXXXXXLXXXXXXWTDXRP

SEQ ID NO: 66 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAEIPAGWXXXXXXXXXXXXLXXXXXXWTDXRP

SEQ ID NO: 67 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAEIPGGWXXXXXXXXXXXXLXXXXXXWTDXRP

SEQ ID NO: 68 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPAFAEIPQGWXXXXXXXXXXXXLXXXXXXWTDXRP

SEQ ID NO: 69 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAEVPDGWXXXXXXXXXXXXLXXXXXXWTDXRP

SEQ ID NO: 70 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAEVPAGWXXXXXXXXXXXXLXXXXXXWTDXRP

SEQ ID NO: 71 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAEVPGGWXXXXXXXXXXXXLXXXXXXWTDXRP

SEQ ID NO: 72 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAEVPQGWXXXXXXXXXXXXLXXXXXXWTDXRP

SEQ ID NO: 73 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAAVPDGWXXXXXXXXXXXXLXXXXXXWTDXRP

SEQ ID NO: 74 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAAVPAGWXXXXXXXXXXXXLXXXXXXWTDXRP

SEQ ID NO: 75 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAAVPGGWXXXXXXXXXXXXLXXXXXXWTDXRP

SEQ ID NO: 76 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAAVPQGWXXXXXXXXXXXXLXXXXXXWTDXRP

SEQ ID NO: 77 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAEIPDGWXXXXXXXXXXXXLXXXXXXWTDXRP

SEQ ID NO: 78 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAEIPAGWXXXXXXXXXXXXLXXXXXXWTDXRP

SEQ ID NO: 79 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAEIPGGWXXXXXXXXXXXXLXXXXXXWTDXRP

SEQ ID NO: 80 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPTFAEIPQGWXXXXXXXXXXXXLXXXXXXWTDXRP

SEQ ID NO: 81 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAEVPDGWXXXXXXXXXXXXLXXXXXXWTDXRP

SEQ ID NO: 82 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAEVPAGWXXXXXXXXXXXXLXXXXXXWTDXRP

SEQ ID NO: 83 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAEVPGGWXXXXXXXXXXXXLXXXXXXWTDXRP

SEQ ID NO: 84 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAEVPQGWXXXXXXXXXXXXLXXXXXXWTDXRP

SEQ ID NO: 85 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAAVPDGWXXXXXXXXXXXXLXXXXXXWTDXRP

SEQ ID NO: 86 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAAVPAGWXXXXXXXXXXXXLXXXXXXWTDXRP

SEQ ID NO: 87 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAAVPGGWXXXXXXXXXXXXLXXXXXXWTDXRP

SEQ ID NO: 88 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAAVPQGWXXXXXXXXXXXXLXXXXXXWTDXRP

SEQ ID NO: 89 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAEIPDGWXXXXXXXXXXXXLXXXXXXWTDXRP

SEQ ID NO: 90 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAEIPAGWXXXXXXXXXXXXLXXXXXXWTDXRP

SEQ ID NO: 91 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAEIPGGWXXXXXXXXXXXXLXXXXXXWTDXRP

SEQ ID NO: 92 - protein, 44 aa, Artificial Sequence
  “Synthetic”
  Synthetic
  SITE 2,4,6,20,21,22,23,24,25,26,27,28,29,30,31,33,34,35,36,37,38,42: Xaa is unknown
NXEXQXSLWPVFAEIPQGWXXXXXXXXXXXXLXXXXXXWTDXRP

(19) United States
(12) Patent Application Publication
(10) Pub. No.: US 2019/0233477 A1
(43) Pub. Date: Aug. 1, 2019

(54) MBTH-LIKE PROTEINS IN EUKARYOTIC NRPS-CATALYZED PROCESSES

(71) Applicants: DSM Sinochem Pharmaceuticals Netherlands B.V., Delft (NL); Rijksuniversiteit Groningen, Groningen (NL)

(72) Inventors: Roelof Ary Lans Bovenberg, Echt (NL); Ulrike Maria Muller, Echt (NL); Arnold Jacob Mathieu Driessen, Groningen (NL); Pohl Carsten, Groningen (NL); Reto Daniel Zwahlen, Groningen (NL)

(73) Assignee: DSM Sinochem Pharmaceuticals Netherlands B.V., Delft (NL)

(21) Appl. No.: 16/093,943

(22) PCT Filed: Apr. 13, 2017
(86) PCT No.: PCT/EP2017/059010

§ 371 Date: Oct. 15, 2018

(30) Foreign Application Priority Data
Apr. 14, 2016 (EP) 16165262.3
Jun. 30, 2016 (EP) 16177069.8

(51) Int. Cl. C07K 14/195; C12P 15/00; C12P 17/02; C12P 35/00; C12P 37/00; C12N 1/14

(52) U.S. Cl. CPC C07K 14/195; C12P 15/00; C12N 1/14; C12P 35/00; C12P 37/00; C12P 17/02

(57) ABSTRACT

The present invention relates to a method to improve the production of a secondary metabolite catalyzed by a non-ribosomal peptide synthetase comprising contacting in a eukaryotic host a eukaryotic non-ribosomal peptide synthetase with an MbtH-like protein. The present invention further relates to a composition comprising a eukaryotic non-ribosomal peptide synthetase that is not a hybrid and a prokaryotic MbtH and to a eukaryotic host cell comprising a non-ribosomal peptide synthetase and a polynucleotide allowing the expression of an MbtH-like protein.

### FIELD OF THE INVENTION

The present invention relates to a method to improve the production of a secondary metabolite catalyzed by a non-ribosomal peptide synthetase comprising contacting in a eukaryotic host a eukaryotic non-ribosomal peptide synthetase with an MbtH-like protein. The present invention further relates to a composition comprising a eukaryotic non-ribosomal peptide synthetase that is not a hybrid and a prokaryotic MbtH and to a eukaryotic host cell comprising a non-ribosomal peptide synthetase and a polynucleotide allowing the expression of an MbtH-like protein.

### BACKGROUND OF THE INVENTION

Secondary metabolites are compounds produced in microorganisms through the modification of primary metabolite synthases. They do not play a role in growth, development, and reproduction like primary metabolites, but many have a role in ecological function, including defense mechanism(s), by serving as antibiotics and by producing pigments. Today, many secondary metabolites have high value for society and are routinely produced on an industrial scale in fermentation processes. Some examples of secondary metabolites with importance in industrial microbiology include atropine, bleomycin and antibiotics such as bacitracin, erythromycin, penicillin and vancomycin.

As is the case with any other production process, also industrial fermentations producing secondary metabolites are the ongoing subject of yield improvement programs. This increases unit productivity, reduces cost and in many cases improves product isolation and purification and thus ultimately, product quality. Next to multitudes of obvious strategies to improve yields of fermentation processes, such as tuning of nutrient compositions, optimizing conditions like pH and temperature, genetically modifying microbial pathways and perfecting downstream processing, there remains an ongoing need to further improve by implementing new technologies that can further stretch productivity.

MbtH-like proteins are small (8-10 kD) proteins with exceptionally conserved sequence motifs resembling MbtH from Mycobacterium tuberculosis. The function of MbtH-like proteins is, to a large extent, still unknown although recent studies indicate a role in the biosynthesis of peptides. The genes encoding MbtH-like proteins, mbtH-like genes, are often found in non-ribosomal peptide synthetase (NRPS) gene clusters. Non-ribosomal peptides (NRP) are an important class of secondary metabolites. Many mbtH-like genes are deposited in GenBank. In order to identify MbtH-like proteins a BLASTP study shows homologues encoded by members of Actinobacteria, Firmacutes and Proteobacteria, however not by Archaea (R. H. Baltz, J. Ind. Microbiol. Biotechnol. (2011) 38, 1747-1760). There are no reports of mbtH-like genes in naturally occurring eukaryotic NRPS gene clusters, their function is exclusively related to prokaryotic NRPS's.

Nevertheless, in WO 2013/113646 the use of an MbtH-like protein in the preparation of semi-synthetic β-lactam antibiotics in Penicillium is described. However, in this case the NRPS is a non-natural hybrid (comprising both eukaryotic and prokaryotic modules) and the MbtH-like protein in question positively influences only the adenylation reaction catalyzed by the prokaryotic module of the NRPS. This confirms the prejudice that MbtH-like proteins only act on prokaryotic NRPS's.

However, eukaryotes (notably fungi like Aspergillus, Penicillium, and Trichoderma) are an important class of microorganisms used in industrial production processes. Several economically attractive secondary metabolites from eukaryotes are for example β-lactam antibiotics, chrysogenins, roquefortins, cyclosporine and echinocandins. They belong to the fungal non-ribosomal petides. The above general need for further improving productivity in industrial fermentations of secondary metabolites equally applies to those processes that are catalyzed by eukaryotic NRPS's.

### DETAILED DESCRIPTION OF THE INVENTION

In the context of the present invention, the term “heterologous” used in combination with modules refers to modules wherein domains, such as adenylation or condensation domains, are from different modules. These different modules may be from the same enzyme or may be from different enzymes.

The term “hybrid” refers to a NRPS that comprises modules from both eukaryotic and prokaryotic origin. Typically a hybrid NRPS is obtained by genetic construction and not naturally occurring. NRPS's that are not hybrid comprise exclusively modules from eukaryotic origin or comprise exclusively modules from prokaryotic origin.

The term “module” defines a catalytic unit that enables incorporation of one peptide building block, usually an amino acid, in the product, usually a peptide, and may include domains for modifications like epimerization and methylation.

The term “non-ribosomal peptide” or “NRP” refers to peptide secondary metabolites, usually produced by microorganisms like bacteria and fungi. NRP's are synthesized by NRPS's. NRP's often have cyclic and/or branched structures, can contain non-proteinogenic amino acids including D-amino acids, carry modifications like N-methyl and N-formyl groups, or are glycosylated, acylated, halogenated, or hydroxylated. Cyclization of amino acids against the peptide “backbone” is often performed, resulting in oxazolines and thiazolines; these can be further oxidized or reduced. On occasion, dehydration is performed on serines, resulting in dehydroalanine. NRP's are often dimers or trimers of identical sequences chained together or cyclized, or even branched. NRP's are often toxins, siderophores, or pigments. Non-ribosomal peptide antibiotics, cytostatics, and immunosuppressants are in commercial use. Examples of NRP's are antibiotics (such as actinomycin, bacitracin, cephalosporin C, daptomycin, gramicidin, penicillin G, penicillin V, teixobactin, tyrocidine, vancomycin, zwittermicin A), antifungals (such as echinocandins or aculeacins), antibiotic precursors (such as ACV tripeptide), cytostatics (such as bleomycin, epothilone), immunosuppressants (such as ciclosporin), nitrogen storage polymers (such as cyanophycin), phytotoxins (such as AM-toxin, HC-toxin), pigments (such as indigoidine), siderophores (such as enterobactin, myxochelin A) and toxins (such as microcystins, nodularins, cyanotoxins)

The term “non-ribosomal peptide synthetase” or “NRPS” refers to a class of modular multi-domain enzymes found in the cytoplasm of bacteria and fungi that synthesize a large variety of highly diverse peptides and which are, unlike ribosomes, independent of messenger RNA. NRPS's are organized in multi-subunit clusters and each subunit in turn is composed of modules, capable of carrying out one cycle of chain elongation. A typical module consists of an adenylation (A) domain, a peptidyl carrier protein (PCP) domain and a condensation (C) domain. A domains (˜550 residues) and C domains (˜450 residues) are responsible for loading PCP domains with the cognate amino acid and catalyzing the peptide bond formation between the upstream aminoacyl or peptidyl PCP and downstream peptidyl PCP, respectively. During the entire process, the growing peptide chain is covalently linked to a phosphopantetheine cofactor which itself is attached to a conserved serine by a dedicated Ppan transferase (Pptase).

The term “secondary metabolite” refers to compounds that are not directly involved in the normal growth, development, or reproduction of an organism. Secondary metabolites are often restricted to a narrow set of species within a phylogenetic group and often play an important role in defense systems. Humans use secondary metabolites as colorings, flavorings and medicines. Examples of secondary metabolites are alkaloids (such as atropine, cocaine, codeine, morphine, tetrodotoxin), natural phenols (such as polyphenols), monoterpenoids (such as geranyl diphosphate, limonene, pinene), diterpenoids (such as aphidicolin, geranylgeranyl diphosphate, pimaradiene, taxol), NRP's, pigments (such as chrysogenin), mycotoxins (such as roquefortin) and antibiotics (such as a β-lactam like 6-aminopenicillanic acid, 7-am inodesacetoxycephalosporanic acid, adipyl-7-aminodesacetoxycephalosporanic acid, cephalosporin C, penicillin G or penicillin V, streptomycin, tetracyclin).

In a first aspect of the invention there is disclosed a method to improve the production of a secondary metabolite or a precursor occurring in the pathway leading to said secondary metabolite catalyzed by a NRPS comprising contacting in a eukaryotic host said NRPS with an MbtH-like protein, characterized in that said NRPS is from eukaryotic origin and is not a hybrid.

Surprisingly it is found that MbtH-like proteins introduced in eukaryotic hosts positively influence the production levels of NRPS-dependent intermediates and other NRPS-dependent secondary metabolites. The present invention demonstrates successful results with a selection of MbtH proteins covering a variety of different sources and grades of homology on the one hand, combined with a range of NRPS's on the other hand that are all fully eukaryotic, i.e. are not hybrids.

Table 1 is a summary of the many examples that have been investigated with three different NRPS's and a range of MbtH-like proteins in two different strains, clearly showing that any combination of the investigated NRPS's and MbtH-like proteins results is a positive effect at least at one point in the pathway of the secondary metabolite.

TABLE 1
Overview of the average number of metabolites effected in
productivity by the presence of MbtH-like proteins from
Tables 4-9 (+: number of metabolites with >10% increased
productivity; +/−: number of metabolites with +/−10%
productivity; −: number of metabolites with >10%
decreased productivity)

| | | Penicillin | | | Chrysogenine | | | Roquefortine | | |
|---|---|---|---|---|---|---|---|---|---|---|
| | | cluster | | | cluster | | | cluster | | |
| Strain | Day | + | +/− | − | + | +/− | − | + | +/− | − |
| DS 17690 | 2 | 1 | 2 | 0 | 9 | 0 | 1 | 2 | 1 | 7 |
| | 5 | 2 | 0 | 1 | 7 | 3 | 0 | 10 | 0 | 0 |
| DS 47274 | 2 | 3 | 0 | 0 | 1 | 6 | 3 | 10 | 0 | 0 |
| | 5 | 3 | 0 | 0 | 5 | 5 | 0 | 10 | 0 | 0 |

In a first embodiment, the eukaryotic host is a fungus or a yeast as in industry these are routinely employed eukaryotic microorganisms. Suitable examples are Aspergillus, Kluyveromyces, Penicillium, Pichia, Saccharomyces, Trichoderma, and Yarrowia and preferably Penicillium chrysogenum, Aspergillus nidulans, Aspergillus niger, Pichia pastoris, Kluyveromyces lactis, Saccharomyces cerevisiae or Yarrowia lipolytica.

In a second embodiment of the invention the secondary metabolite is as defined above, is preferably a β-lactam, a pigment or a mycotoxin. More preferably, the β-lactam is 6-aminopenicillanic acid, 7-aminodesacetoxycephalosporanic acid, adipyl 7-aminodesacetoxycephalosporanic acid, cephalosporin C, penicillin G or penicillin V, the pigment is a chrysogenin and the mycotoxin is a roquefortin.

In a third embodiment the eukaryotic host strains are high level production strains. It has been found that e.g. high level penicillin production in some strains of Penicillium chrysogenum is due to presence of amplified tandem repeats of the penicillin gene cluster (reviewed in Martin F. (2000) J. Bacteriol 182:2355-2362.). High level production of secondary metabolites can be the result of amplified biosynthesis gene clusters and so the eukaryotic host is a multi copy strain with respect to the secondary metabolite cluster of interest.

In a fourth embodiment, preferred MbtH-like proteins are the ones described in R. H. Baltz (J. Ind. Microbiol. Biotechnol. (2011) 38, 1747-1760). More preferred MbtH-like proteins are the ones comprising invariant amino acids N17, E19, Q21, S23, W25, P26, P32, G34, W35, L48, W55, T56, D57, R59 and P60, also suitably referred to with the amino acid code NXEXQXSXWP-X₅-PXGW-X₁₂-L-X₆-WTDXRP (SEQ ID NO: 17). In the above annotation the letters D, E, G, L, N, P, Q, R, S, T, W and X refer to the commonly known single letter codes for amino acids (whereby X denotes one unspecified amino acid, X₅ denotes 5 unspecified amino acids, X₆ denotes 6 unspecified amino acids and X₁₂ denotes 12 unspecified amino acids). In preferred embodiments said MbtH-like protein comprises the amino acid code NXEXQXSXWP-X₅-PDGW-X₁₂-L-X₆-WTDXRP or NXEXQXSXWP-X₅-PAGW-X₁₂-L-X₆-WTDXRP or NXEXQXSXWP-X₅-PGGW-X₁₂-L-X₆-WTDXRP or NXEXQXSXWP-X₅-PQGW-X₁₂-L-X₆-WTDXRP wherein X₅ is chosen from the list consisting of AFAEV, AFAAV, AFAEI, TFAEV, TFAAV, TFAEI, VFAEV, VFAAV and VFAEI (SEQ ID NO: 18-SEQ ID NO: 53). In more preferred embodiments said MbtH-like protein comprises the amino acid code NXEXQXSLWP-X₅-PDGW-X₁₂-L-X₆-WTDXRP or NXEXQXSLWP-X₅-PAGW-X₁₂-L-X₆-WTDXRP or NXEXQXSLWP-X₅-PGGW-X₁₂-L-X₆-WTDXRP or NXEXQXSLWP-X₅-PQGW-X₁₂-L-X₆-WTDXRP wherein X₅ is chosen from the list consisting of AFAEV, AFAAV, AFAEI, TFAEV, TFAAV, TFAEI, VFAEV, VFAAV and VFAEI (SEQ ID NO: 57-SEQ ID NO: 92).

It is noted that in R. H. Baltz (J. Ind. Microbiol. Biotechnol. (2011) 38, 1747-1760) and in WO 2013/113646 erroneously the amino acid code NXEXQXSXWP-X₅-PXGW-X₁₃-L-X₇-WTDXRP is mentioned where in fact this refers to and should be NXEXQXSXWP-X₅-PXGW-X₁₂-L-X₆-WTDXRP. Preferably, the MbtH-like proteins of the present invention are Tcp13 (SEQ ID NO: 1) or Tcp17 (SEQ ID NO: 2) obtained from the teicoplanin biosynthesis cluster from Actinoplanes teichomyceticus (Sosio et. al., Microbiology (2004) 150, 95-102), or the MbtH-like homologue identified in the Veg biosynthesis cluster obtainable from an uncultured soil bacterium (Banik J. J. and Brady S. F., Proc. Natl. Acad. Sci. USA (2008) 105, 17273-17277) encoded by nt 33826-34035 of GenBank: EU874252 (SEQ ID NO: 3) called VEG8 or the MbtH-like homologue identified in the Teg biosynthesis cluster obtainable from an uncultured soil bacterium (Banik J. J. and Brady S. F., Proc. Natl. Acad. Sci. USA (2008) 105, 17273-17277) encoded by nt 33949-33158 of GenBank: EU874253 (SEQ ID NO: 4) called TEG or the MbtH-like homologue (SEQ ID NO: 5) identified in the balhimycin biosynthesis cluster from Amycolatopsis balhimycina (Recktenwald et al., Microbiology (2002) 148, 1105-1118, Stegman et al., FEMS Microbial Lett. (2006) 262, 85-92) called BPS or the MbtH-like homologue (SEQ ID NO: 6) identified in the complestatine biosynthesis cluster from Streptomyces lavendulae (Chiu et al., Proc. Natl. Acad. Sci. USA (2001) 98, 8548-8553) called COM or MbtH like homologue SCO0489 (SEQ ID NO: 7) identified in the calcium dependent antibiotic (CDA) biosynthesis cluster from Streptomyces coelicolor (Hojati et al. (Chem. & Biol. (2002) 9, 1175-1187) called CDAI or MbtH-like proteins having an amino sequence with a percentage identity of at least 70%, more preferably at least 80%, even more preferably at least 90%, most preferably at least 95% to said sequences. Such polypeptide modules with a percentage identity of at least 70% are also called homologous sequences or homologues.

In a second aspect the invention provides a composition comprising a eukaryotic NRPS that is not a hybrid and a prokaryotic MbtH. Preferred combinations of eukaryotic NRPS's and MbtH-like proteins are the NRPS N-(5-amino-5-carboxypentanoyl)-L-cysteinyl-D-valine synthase PcbAB (UniProtKB—P26046 (ACVS2_PENCH, SEQ ID NO: 54) with COM, PcbAB with CDAI, PcbAB with TCP13, PcbAB with TEG, PcbAB with VEG8, the NRPS Pc21g12630 protein ChyA (UniProtKB—B6HLP9 (B6HLP9_PENRW, SEQ ID NO: 55) with COM, ChyA with CDAI, ChyA with TCP13, ChyA with TEG, ChyA with VEG8, the NRPS Roquefortine/meleagrin synthesis protein A RogA (UniProtKB—B6HJU6.1 ROQA_PENRW, SEQ ID NO: 56) with COM, RogA with CDAI, RogA with TCP13, RogA with TEG or RogA with VEG8. Thus, preferred combinations are SEQ ID NO: 54 with SEQ ID NO: 1, SEQ ID NO: 54 with SEQ ID NO: 3, SEQ ID NO: 54 with SEQ ID NO: 4, SEQ ID NO: 54 with SEQ ID NO: 6, SEQ ID NO: 54 with SEQ ID NO: 7, SEQ ID NO: 55 with SEQ ID NO: 1, SEQ ID NO: 55 with SEQ ID NO: 3, SEQ ID NO: 55 with SEQ ID NO: 4, SEQ ID NO: 55 with SEQ ID NO: 6, SEQ ID NO: 55 with SEQ ID NO: 7, SEQ ID NO: 56 with SEQ ID NO: 1, SEQ ID NO: 56 with SEQ ID NO: 3, SEQ ID NO: 56 with SEQ ID NO: 4, SEQ ID NO: 56 with SEQ ID NO: 6, SEQ ID NO: 56 with SEQ ID NO: 7, or sequences that are at least 90% homologous to any or both.

In a third aspect the invention provides a eukaryotic host cell comprising a NRPS from eukaryotic origin that is not a hybrid and a polynucleotide allowing the expression of an MbtH-like protein. Preferably the MbtH-like proteins are those of the first aspect of the invention. Preferably the host cell is a fungus or a yeast. Suitable examples are Aspergillus, Kluyveromyces, Penicillium, Pichia, Saccharomyces, Trichoderma, and Yarrowia and preferably Penicillium chrysogenum, Aspergillus nidulans, Aspergillus niger, Pichia pastoris, Kluyveromyces lactis, Saccharomyces cerevisiae or Yarrowia lipolytica. Preferably the host cell comprises an MbtH-like protein with SEQ ID NO: 17.

In a fourth aspect the invention provides a method for the preparation of the host cell of the third aspect of the invention. This may be achieved according to procedures known to the skilled person such as targeted or random integration of an expression cassette consisting of a suitable promoter, the gene of interest and a terminator.

Throughout this description the following three letter codes and one letter codes are used for amino acids:

| Amino acid | Three letter code | One letter code |
|---|---|---|
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamic acid | Glu | E |
| Glutamine | Gln | Q |
| Glutamine or glutamic acid | Glx | Z |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any/unknown | Xaa | X |

### Homology & Identity

The terms “homology” or “percent identity” are used interchangeably herein. For the purpose of this invention, it is defined here that in order to determine the percent homology of two amino acid sequences or of two nucleic acid sequences, the sequences are aligned for optimal comparison purposes. In order to optimize the alignment between the two sequences gaps may be introduced in any of the two sequences that are compared. Such alignment can be carried out over the full length of the sequences being compared. Alternatively, the alignment may be carried out over a shorter length, for example over about 20, about 50, about 100 or more nucleic acids/based or amino acids. The identity is the percentage of identical matches between the two sequences over the reported aligned region.

A comparison of sequences and determination of percent identity between two sequences can be accomplished using a mathematical algorithm. The skilled person will be aware of the fact that several different computer programs are available to align two sequences and determine the homology between two sequences (Kruskal, J. B. (1983) An overview of sequence comparison In D. Sankoff and J. B. Kruskal, (ed.), Time warps, string edits and macromolecules: the theory and practice of sequence comparison, pp. 1-44 Addison Wesley). The percent identity between two amino acid sequences may be determined using the Needleman and Wunsch algorithm for the alignment of two sequences. (Needleman, S. B. and Wunsch, C. D. (1970) J. Mol. Biol. 48, 443-453). The algorithm can align both amino acid sequences and nucleotide sequences. The Needleman-Wunsch algorithm has been implemented in the computer program NEEDLE. For the purpose of this invention the NEEDLE program from the EMBOSS package was used (version 2.8.0 or higher, EMBOSS: The European Molecular Biology Open Software Suite (2000) Rice, P. Longden, I. and Bleasby, A. Trends in Genetics 16, (6) pp 276-277, http://emboss.bioinformatics.nl/). For protein sequences, EBLOSUM62 is used for the substitution matrix. For nucleotide sequences, EDNAFULL is used. Others can be specified. The optional parameters used are a gap-open penalty of 10 and a gap extension penalty of 0.5. The skilled person will appreciate that all these different parameters will yield slightly different results but that the overall percentage identity of two sequences is not significantly altered when using different algorithms.

Global Homology Definition

The homology or identity is the percentage of identical matches between the two sequences over the total aligned region including any gaps. The homology or identity between the two aligned sequences is calculated as follows: Number of corresponding positions in the alignment showing an identical amino acid in both sequences divided by the total length of the alignment including the gaps. The identity defined as herein can be obtained from NEEDLE and is labelled in the output of the program as “IDENTITY”.

Longest Identity Definition

The homology or identity between the two aligned sequences is calculated as follows: Number of corresponding positions in the alignment showing an identical amino acid in both sequences divided by the total length of the alignment after subtraction of the total number of gaps in the alignment. The identity defined as herein can be obtained from NEEDLE by using the NOBRIEF option and is labelled in the output of the program as “longest-identity”.

### EXAMPLES

### General Materials and Methods

Molecular and Genetic Techniques

Standard genetic and molecular biology techniques are known in the art (e.g. Maniatis et al. “Molecular cloning: a laboratory manual” (1982) Cold Spring Harbor Laboratory, Cold Spring Harbor, N.Y.; Miller “Experiments in molecular genetics” (1972) Cold Spring Harbor Laboratory, Cold Spring Harbor, N.Y.; Sambrook and Russell “Molecular cloning: a laboratory manual” (3rd edition)” (2001) Cold Spring Harbor Laboratory, Cold Spring Harbor Laboratory Press; Ausubel “Current protocols in molecular biology” (1987) Green Publishing and Wiley Interscience, New York).

Plasmids and Strains

Escherichia coli:

Cloning was performed using Escherichia coli DH5a.

Penicillium chrysogenum:

DS 17690 (Harris D. et al., Metab. Eng. (2006) 8, 91-101) was used as a high level penicillin production strain of Penicillium chrysogenum, DS 47274 (Harris D. et al., Metab. Eng. (2006) 8, 91-101) was used as a low level penicillin production strain. They differ in the number of penicillin biosynthetic gene clusters. While the original strain DS17690 contains 8 copies, DS47274 is a one copy strain (Harris D. et al., Metab. Eng. (2006) 8, 91-101).

Media:

Escherichia coli:

All cultures were grown using 2×PY (15 g/L Bacto-tryptone, 10 g/L Yeast extract, 10 g/L Sodium chloride, pH 7.0) at 37° C. and 200 rpm. Antibiotics (25 μg/mL Zeocin) were supplemented to maintain plasmid for pIAT.

Penicillium chrysogenum:

Penicillium chrysogenum strains were grown on YGG medium (Bartoszewska M. et al., Appl. Environ. Microbiol. (2011) 77, 1413-14221.

Sporulation of mycelia was stimulated by growth on R agar (Bartoszewska M. et al., Appl. Environ. Microbiol. (2011) 77, 1413-14221) at 25° C. for 10-11 days.

For genetic manipulation purposes,

Transformants were selected as cotransformants by their ability to grow on plates with acetamide as the only nitrogen source. Acetamide-agar comprises 0.04 mM FeSO₄, 2.03 mM MgSO₄, 51.3 mM NaCl, 55.5 mM Glucose, 15 g/l Agar Agar, 10 g/l Acetamide, 5.1 mM, KH₂PO₄, 4.9 mM K₂HPO₄, and 2 ml/l Trace element solution (Na₃C₆H₅O₇, 149 mM, FeSO₄, 89 mM, MgSO₄, 1.04 mM, H₃BO₃, 0.2 mM, Na₂MoO₄, 0.05 mM, CuSO₄, 2.56 mM, ZnSO₄, 8.76 mM, CoSO₄, 2.28 mM, MnSO₄, 17.99 mM, CaCl₂, 10.88 mM, EDTA, 107 mM).

For production of secondary metabolites, penicillin production medium, PPM (Nijland J. G. et al., Appl. Environ. Microbiol. (2010) 76, 7109-7115)+0.05% phenoxyacetic acid. was used.

### Example 1

### Synthetic Design and Cloning of MbtH-Like Protein Expression Constructs

To express the MbtH-like proteins in Penicillium chrysogenum, expression cassettes comprising the Penicillium chrysogenum IPNS promoter and the Penicillium chrysogenum AT terminator were designed. The vector pIAT comprising the promoter cassette from the isopenicillin N synthetase (IPNS, pcbC-gene) of Penicillium chrysogenum (Promoter pcbC) flanked by NotI/NdeI sites (SEQ ID No 8), a DNA fragment harbouring a cat-ccdB cassette (Chloramphenicol resistance and a toxicity gene for Escherichia coli), and a transcription terminator cassette from the acyl-CoA:isopenicillin N acyltransferase (AT, pcbDE-gene) of Penicillium chrysogenum flanked by NsiI/NotI-sites (SEQ ID No 9) was used for cloning of the MbtH encoding genes as NdeI/NsiI fragments between promoter and terminator.

Five different MbtH-like proteins were chosen, one from the teicoplanin biosynthetic cluster annotated as tcp13 (SEQ ID NO: 1, GenBank: AJ605139 Genomic DNA; Translation: CAE53354.1) and called TCP13, one from the Veg biosynthetic clusters identified by a search for homologous MbtH-like sequences in the Veg Cluster (SEQ ID NO: 3, GenBank: EU874252, nt 33826-34035, between veg9 and veg10), called VEG8, one from the complestatine biosynthetic cluster annotated as hypothetical protein (SEQ ID NO: 6, GenBank: AF386507 Genomic DNA; Translation: AAK81828.1) and called COM, one from the Teg biosynthetic clusters identified by a search for homologous MbtH-like sequences in the Teg Cluster (SEQ ID NO: 4, GenBank: EU874253, nt 32949-33158, between teg8 and teg9), called TEG, and one from the calcium dependent antibiotic biosynthesis cluster, SC00489 (SEQ ID NO: 7) from Streptomyces coelicolor (Hojati et al. (Chem. & Biol. (2002) 9, 1175-1187) called CDAI. Target genes encoding the selected proteins were constructed synthetically resulting in nucleotide SEQ ID NO: 10-14 and ordered at IDT as gBlocks (Integrated DNA technology, Coralville, Iowa, USA) flanked by restriction sites NdeI and NsiI for final cloning between IPNS promoter and AT terminator. The gene encoding VEG8 was used as wild type sequence, while the genes encoding TCP13, COM, CDAI, BPS and TEG were codon optimized for expression in Penicillium chrysogenum.

The final plasmids harbouring the expression constructs for overexpression of the MbtH-like proteins in Penicillium chrysogenum constructed by cloning the NdeI/NsiI fragments taken from the gBlocks into the NdeI/NsiI sites of expression vector pIAT were named pIAT-Tcp13, pIAT-Veg8, pIAT-COM, pIAT-TEG, and pIAT-CDAI. The final sequences of the MbtH expression constructs were confirmed using sequencing provided by Macrogen (Macrogen Europe, Amsterdam, The Netherlands)

### Example 2

### Transformation of MbtH-Like Protein Expression Constructs

pIAT-MbtH plasmids were cut with NotI, and run on an agarose gel. The NotI-fragments comprising the MbtH expression cassettes were cut from the agarose gels, and gel-cut fragments were purified and concentrated by desalting. Protoplast formation and transformations of two Penicillium chrysogenum strains, DS 17690 and DS 47274, was performed as described by Kovalchuk A et al. Methods Mol Biol. (2012); 835: 1-16.

Transformation was performed as co-transformation with the amdS selection marker comprising the Aspergillus nidulans acetamidase encoding gene amdS under control of the Aspergillus nidulans gpdA promoter (U.S. Pat. No. 5,876,988, Selten G C M, Swinkels B W, van Gorcom R F M. 1999. Selection marker gene free recombinant strains: method for obtaining them and the use of these strains) in a molar ratio of 10:1 (MbtH expression construct: amdS selection Marker). This transformation approach results in random integration of the MbtH expression construct and the amdS selection marker in the genome of the host organisms and the number of expression cassettes can vary per transformant obtained.

Because of this variation in most cases we have investigated multiple independent transformants per host strain/MbtH expression construct variation instead of limiting ourselves to one.

After 12 days, several selected colonies for each MbtH/amdS co-transformation were purified using three acetamide-agar to R-agar plate transfer cycles. During purification, colonies exhibiting insufficient growth on selective acetamide agar or too fast sporulation on R-agar-plates, were discarded, respectively.

To confirm MbtH integration, pieces of mycelium from selected colonies were homogenized in milliQ water and used as template DNA in a PCR reaction setup. Primers were targeting the 5′ flanking IPNS promoter (SEQ ID No: 15) and the 3′ AT terminator (SEQ ID No: 16) of each MbtH expression cassette.

Finally, three to nine amdS positive and MbtH expression construct containing transformants per MbtH expression construct and strain background were obtained for further characterization, with the exception of expression construct VEG8; here one transformant only for strain background DS17690 and two transformants for strain background DS47274 were obtained. Table 2 gives an overview on the transformants obtained and the codes chosen for the different transformants.

TABLE 2
Overview on amdS positive and MbtH gene containing transformants
for high and low level penicillin producing Penicillium chrysogenum
strains DS17690 and DS47274.

strain background

| MbtH expressed | DS17690 | DS47274 |
|---|---|---|
| COM | 1-2 | 7-3 |
| | 1-3 | Com_IV(+) |
| | 1-5 | Com_XIV(+) |
| | 1-6 | Com_XII(+) |
| | 2-2 | 8-1 |
| | 2-3 | 8-4 |
| | 2-4 | Com_VIII(+) |
| | | Com_II(+) |
| | | Com_XI(+) |
| CDAI | 3-1 | 9-2 |
| | 3-2 | 9-3 |
| | 3-3 | 9-4 |
| | 3-4 | 9-5 |
| TCP13 | 4-1 | 10-2 |
| | 4-2 | 10-5 |
| | 4-3 | 10-6 |
| | 4-4 | |
| | Tcp13_II(+) | |
| | Tcp13_III(+) | |
| TEG | 5-1 | 11-2 |
| | 5-2 | 11-3 |
| | 5-4 | 11-5 |
| | 5-5 | |
| | Teg_II(+) | |
| VEG8 | 6-3 | 12-4 |
| | | Veg8_XI(+) |

### Example 3

### Small Scale Fermentations of MbtH-Like Protein Expressing Penicillium chrysogenum Strains for Secondary Metabolite Production

Positively tested Penicillium strains as summarized in Table 2 were subjected to small scale fermentation experiments in 100 ml shake flasks over a total course of 5 days. As reference strains, the non transformed strains DS17690 and DS47274 were taken along. Cultures were inoculated from spore crops in a volume of 25 mL YGG medium. After 24 h of growth, the sporulated pre-culture was 10-fold diluted in a total volume of 2×30 mL in PPM plus 0.25% phenoxyacteic acid. Two biological and two technical replicates were used per strain and experiment. All cultures were grown at 250° C. and 200 rpm using Innova 44 shaker (Eppendorf, Hamburg, Germany). Sampling was conducted at day 2 and 5 after pre-culture transfer. Thereby, 1 mL of culture was taken, centrifuged at 40° C., 14000×g for 10 minutes. The supernatant is subsequently filtered using a PTFE syringe filter (0.2 μM, No. 514-0068, VWR, Radnor, Pa., USA), reduced with 10 mM DTT (1,4-Dithiotreithol, No. 6908.2, Carl Roth GmbH, Karlsruhe, Germany) and stored at −80° C. up until analysis. The remaining volume of the culture after 5 days is additionally used in order to determine the dry weight.

### Example 4

### Analysis of Secondary Metabolite Production

The genome of Penicillium chrysogenum encodes ten NRPS, twenty polyketide synthases (PKS), and two hybrid NRPS-PKS genes (van den Berg et al. (Nature Biotech (2008) 26, 1161-1168). Several of these genes have been associated with specific secondary metabolites. Three groups of secondary metabolites for which biosynthesis routes have been assigned to specific NRPSs were chosen for analysis of secondary metabolite production: Penicillin related secondary metabolites (NRPS: PcbAB—Pc21g21390), Roquefortine related metabolites (RoqA—Pc21g15480), and Chrysogine related metabolites (NRPS: ChyA—Pc21g12630), respectively. Available standards were used to identify peaks according to retention time and accurate mass by LC/MS analyses. A total of 26 metabolites are associated with these three clusters (Salo O. V., BMC Genomics (2015), 16:937). Table 3 gives an overview on the detectable reference compounds by the LC/MS method applied. The biosynthesis cluster, compound names, monoisotopic ionized masses (M/Z [H]+), molecular composition and retention times for the applied LC program are indicated.

In the culture samples, 23 out of the 26 metabolites were sufficiently abundant, to allow for comparison with the wild type strain. The 23 relevant metabolites can be assigned to the three clusters in the following manner; 3 metabolites for Penicillin biosynthesis, 10 metabolites for Chrysogine biosynthesis, and 10 metabolites for Roquefortine biosynthesis. Metabolite levels were further evaluated by measuring the peak area, normalizing for dry weight and finally calculating ratios, relative to wildtype metabolite abundance. A complete list of all metabolites and intermediates which were identified in the MbtH expressing transformants and their relative abundance compared to the untransformed wildtype strains is summarized in Tables 4-9, whereby Table 4, Table 5 and Table 6 show relative productivity of secondary metabolites in the penicillin, roquefortin and chrysogine cluster, respectively, in high level penicillin production strain DS17690 in the presence of the MbtH-like proteins investigated after 2 and 5 days of cultivation, while Tables 7-9 show this for the low level penicillin production strain DS47274. For each metabolite of the respective cluster measured at the same cultivation time point and for the same strain background, the average of the relative productivity for all MbtH expressing transformants was calculated to visualize the observed trends. Metabolites were classified as a) increased, when the average value was above 1.1 (>10% increased productivity), b) decreased, when the average value was below 0.9 (>10% decreased productivity), and as unchanged, when the average value x was 1.1≥x≥0.9 (+/−10% productivity). Finally, the total number of metabolites classified as increased, decreased or unchanged per biosynthesis cluster, strain and cultivation day was determined and is given as overall summary in Table 1.

5 μL of every culture supernatant sample obtained from Penicillium fermentations were subjected to LC/MS analysis. Two technical replicates were run per sample. Analysis was performed using a LC/MS Orbitrap machine (Thermo Scientific) in combination with a RP-C18 column (Shimadzu Shim pack XR-ODS 2.2; 3.0×75 mm) in positive mode. A gradient program with MiliQ water (A), Acetonitrile (B) and 2% Formic acid (D) was run; 0 min; A 90%, B 5%, C 5%; 4 min, A 90%, B 5%, C 5%; 13 min, A 0%, B 95%, C 5%; 16 min A 0%, B 95%, C 5%; 16 min, A 90%, B 5%, C 5%; 21 min A 90%, B 5%, C 5% at a flow rate of 0.3 ml min⁻¹.

### Legend to Tables 3-9

Table 3: Overview on secondary metabolites and their corresponding biosynthetic pathways measured with the applied LC program.
Table 4: Relative productivity of secondary metabolites in the penicillin cluster in strain DS17690 in the presence of MbtH-like proteins indicated after 2 and 5 days of cultivation. Productivity is compared to the non modified strain DS17690 (without presence of MbtH-like proteins), for which all values are set to 1.0.
Table 5a: Relative productivity of secondary metabolites in the chrysogenin cluster in strain DS17690 in the presence of MbtH-like proteins after 2 days of cultivation. Productivity is compared to the non modified strain DS17690 (without presence of MbtH-like proteins), for which all values are set to 1.0.
Table 5b: Relative productivity of secondary metabolites in the chrysogenin cluster in strain DS17690 in the presence of MbtH-like proteins after 5 days of cultivation. Productivity is compared to the non modified strain DS17690 (without presence of MbtH-like proteins), for which all values are set to 1.0.
Table 6a: Relative productivity of secondary metabolites in the roquefortine cluster in strain DS17690 in the presence of MbtH-like proteins after 2 days of cultivation. Productivity is compared to the non modified strain DS17690 (without presence of MbtH-like proteins), for which all values are set to 1.0.
Table 6b: Relative productivity of secondary metabolites in the roquefortine cluster in strain DS17690 in the presence of MbtH-like proteins after 5 days of cultivation. Productivity is compared to the non modified strain DS17690 (without presence of MbtH-like proteins), for which all values are set to 1.0.
Table 7: Relative productivity of secondary metabolites in the penicillin cluster in strain DS47274 in the presence of MbtH-like proteins after 2 and 5 days of cultivation. Productivity is compared to the non modified strain DS47274 (without presence of MbtH-like proteins), for which all values are set to 1.0.
Table 8a: Relative productivity of secondary metabolites in the chrysogenin cluster in strain DS47274 in the presence of MbtH-like proteins after 2 days of cultivation. Productivity is compared to the non modified strain DS47274 (without presence of MbtH-like proteins), for which all values are set to 1.0.
Table 8b: Relative productivity of secondary metabolites in the chrysogenin cluster in strain DS47274 in the presence of MbtH-like proteins after 5 days of cultivation. Productivity is compared to the non modified strain DS47274 (without presence of MbtH-like proteins), for which all values are set to 1.0.
Table 9a: Relative productivity of secondary metabolites in the roquefortine cluster in strain DS47274 in the presence of MbtH-like proteins after 2 days of cultivation. Productivity is compared to the non modified strain DS47274 (without presence of MbtH-like proteins), for which all values are set to 1.0.
Table 9b: Relative productivity of secondary metabolites in the roquefortine cluster in strain DS47274 in the presence of MbtH-like proteins after 5 days of cultivation. Productivity is compared to the non modified strain DS47274 (without presence of MbtH-like proteins), for which all values are set to 1.0.

TABLE 3

| | | M/Z | | RT |
|---|---|---|---|---|
| Cluster | Compound | [H]⁺ | Formula | (min) |
| Penicillin | 6-Aminopenicillanic | 217.06 | C₈H₁₂N₂O₃S | 1.84 |
| | acid | | | |
| | δ-(L-α-Amino- | 364.15 | C₁₄H₂₅N₃O₆S | 7.80 |
| | adipyl)-L-cysteinyl- | | | |
| | D-valine | | | |
| | Isopenicillin N | 360.12 | C₁₄H₂₁N₃O₆S | 2.44 |
| | Penicillin G | 335.11 | C₁₆H₁₈N₂O₄S | 10.47 |
| Chrysogine | N-pyrovoyl- | 207.08 | C₁₀H₁₀N₂O₃ | 7.48 |
| | anthranilamid | | | |
| | Chrysogine VII | 277.08 | C₁₃H₁₂N₂O₅ | 7.94 |
| | Chrysogine XI | 338.13 | C₁₅H₁₉N₃O₆ | 8.17 |
| | Chrysogine XII | 337.15 | C₁₅H₂₀N₄O₅ | 7.07 |
| | Chrysogine XIII | 295.11 | C₁₃H₁₄N₂O₆ | 7.95 |
| | Chrysogine XIV | 276.10 | C₁₃H₁₃N₃O₄ | 8.28 |
| | Chrysogine XV | 336.11 | C₁₅H₁₈N₃O₆ | 9.43 |
| | Chrysogine XVI | 413.15 | C₂₀H₂₀N₄O₆ | 8.90 |
| | Chrysogine | 191.08 | C₁₀H₁₀N₂O₂ | 8.24 |
| | Chrysogine B | 250.12 | C₁₃H₁₅N₃O₃ | 7.99 |
| | Chrysogine C | 294.11 | C₁₃H₁₅N₃O₅ | 7.95 |
| Roquefortine | Histidyltrypto- | 324.15 | C₁₇H₁₇N₅O₂ | 5.51 |
| | phanyldi- | | | |
| | ketopiperazine | | | |
| | (HTD) | | | |
| | Dehydrohistidyl- | 322.13 | C₁₇H₁₅N₅O₂ | 6.62 |
| | tryptophanyldiketo- | | | |
| | piperazine (DHTD) | | | |
| | Roquefortine C | 390.19 | C₂₂H₂₃N₅O₂ | 9.49 |
| | Roquefortine D | 392.21 | C₂₂H₂₅N₅O₂ | 8.94 |
| | Roquefortine F | 420.20 | C₂₃H₂₅N₅O₃ | 9.75 |
| | Roquefortine M | 422.18 | C₂₂H₂₃N₅O₄ | 8.92 |
| | Roquefortine N | 440.19 | C₂₂H₂₅N₅O₅ | 8.19 |
| | Glandicoline A | 404.17 | C₂₂H₂₁N₅O₃ | 9.72 |
| | Glandicoline B | 420.17 | C₂₂H₂₁N₅O₄ | 8.93 |
| | Meleagrine | 434.18 | C₂₃H₂₃N₅O₄ | 9.17 |
| | Neoxaline | 436.19 | C₂₃H₂₆N₅O₄ | 9.17 |

TABLE 4

| | | Day 2 | | | Day 5 | | |
|---|---|---|---|---|---|---|---|
| MbtH transformant | | 6-Aminopenicillanic acid | LLD-ACV | Penicillin G | 6-Aminopenicillanic acid | LLD-ACV | Penicillin G |
| COM | 1-2 | 0.39 | 0.90 | 1.69 | 1.81 | 0.52 | 0.68 |
| | 1-3 | 0.21 | 0.93 | 1.99 | 1.73 | 0.57 | 2.90 |
| | 1-5 | 0.18 | 0.84 | 1.54 | 2.78 | 0.41 | 2.85 |
| | 1-6 | 0.45 | 0.68 | 1.42 | 2.17 | 0.26 | 1.30 |
| | 2-2 | 0.51 | 0.88 | 1.83 | 2.09 | 0.13 | 3.02 |
| | 2-3 | 0.90 | 1.52 | 1.75 | 2.62 | 0.29 | 3.22 |
| | 2-4 | 0.75 | 0.70 | 1.99 | 1.90 | 0.71 | 2.90 |
| CDAI | 3-1 | 0.56 | 0.58 | 1.10 | 0.88 | 0.09 | 1.57 |
| | 3-2 | 0.94 | 1.07 | 1.57 | 2.41 | 1.06 | 2.70 |
| | 3-3 | 1.17 | 1.38 | 1.73 | 2.39 | 1.44 | 2.18 |
| | 3-4 | 1.09 | 0.75 | 1.62 | 2.31 | 0.38 | 2.15 |
| TCP13 | 4-1 | 0.12 | 0.11 | 2.06 | 0.28 | 0.15 | 0.84 |
| | 4-2 | 0.92 | 0.72 | 1.56 | 1.68 | 0.30 | 1.90 |
| | 4-3 | 0.83 | 1.10 | 1.82 | 1.49 | 0.74 | 1.91 |
| | 4-4 | 1.15 | 1.50 | 2.54 | 2.47 | 1.20 | 3.39 |
| | Tcp13_II(+) | 2.21 | 0.61 | 1.41 | 1.40 | 2.49 | 1.13 |
| | Tcp13_III(+) | 1.52 | 0.33 | 1.08 | 1.12 | 1.73 | 0.87 |
| TEG | 5-1 | 0.39 | 0.54 | 2.14 | 0.67 | 0.41 | 1.55 |
| | 5-2 | 0.98 | 1.07 | 1.86 | 1.37 | 0.18 | 1.90 |
| | 5-4 | 1.09 | 0.53 | 2.13 | 1.86 | 0.15 | 1.77 |
| | 5-5 | 0.93 | 1.09 | 1.73 | 1.45 | 0.25 | 1.67 |
| | Teg_II(+) | 4.73 | 4.79 | 1.05 | 1.31 | 2.30 | 0.90 |
| VEG8 | 6-3 | 1.09 | 1.38 | 1.62 | 1.58 | 0.81 | 1.34 |

| | | | | | | |
|---|---|---|---|---|---|---|
| All MbtH Avg. | 1.00 | 1.04 | 1.71 | 1.73 | 0.72 | 1.94 |

TABLE 5a

| MbtH transformant | | Chrysognie | Chrysogie B | Chrysogine C | Chrysoginie 7 | Chrysogini 11 | Chrysogenin 12 |
|---|---|---|---|---|---|---|---|
| COM | 1-2 | 1.52 | 2.03 | 1.17 | 1.02 | 1.18 | 1.41 |
| | 1-3 | 0.70 | 1.92 | 1.08 | 0.99 | 0.90 | 1.03 |
| | 1-5 | 1.26 | 5.02 | 1.35 | 1.35 | 1.15 | 1.37 |
| | 1-6 | 1.67 | 6.03 | 1.12 | 1.09 | 0.99 | 1.05 |
| COM | 2-2 | 1.55 | 5.51 | 1.41 | 1.30 | 1.27 | 1.75 |
| | 2-3 | 1.34 | 1.15 | 1.37 | 1.24 | 1.41 | 1.56 |
| | 2-4 | 1.18 | 2.80 | 0.97 | 0.87 | 0.69 | 0.75 |
| CDAI | 3-1 | 1.10 | 2.70 | 1.05 | 0.93 | 1.03 | 1.29 |
| | 3-2 | 1.18 | 1.48 | 1.33 | 1.19 | 1.09 | 1.14 |
| | 3-3 | 1.30 | 1.15 | 1.18 | 1.05 | 0.89 | 1.05 |
| | 3-4 | 0.83 | 1.39 | 1.21 | 1.09 | 1.18 | 1.07 |
| TCP13 | 4-1 | 0.48 | 0.57 | 1.71 | 1.58 | 0.38 | 0.81 |
| | 4-2 | 1.29 | 1.87 | 1.47 | 1.36 | 1.56 | 1.64 |
| | 4-3 | 1.44 | 1.60 | 1.41 | 1.31 | 1.50 | 1.85 |
| | 4-4 | 1.59 | 2.22 | 1.85 | 1.71 | 1.65 | 2.17 |
| | Tcp13_II(+) | 2.24 | 1.57 | 1.35 | 1.35 | 1.98 | 1.79 |
| | Tcp13_III(+) | 1.33 | 1.03 | 0.96 | 0.96 | 1.23 | 1.18 |
| TEG | 5-1 | 1.36 | 2.15 | 1.48 | 1.38 | 1.80 | 2.42 |
| | 5-2 | 1.36 | 1.99 | 1.45 | 1.35 | 1.52 | 1.99 |
| | 5-4 | 1.10 | 2.21 | 1.67 | 1.56 | 1.86 | 2.01 |
| | 5-5 | 1.27 | 1.65 | 1.41 | 1.32 | 1.67 | 1.89 |
| | Teg_II(+) | 2.52 | 2.33 | 1.32 | 1.34 | 3.31 | 3.25 |
| VEG8 | 6-3 | 1.22 | 1.87 | 1.51 | 1.44 | 1.81 | 1.95 |

| | | | | | | |
|---|---|---|---|---|---|---|
| All MbtH Avg. | 1.34 | 2.27 | 1.34 | 1.25 | 1.39 | 1.58 |

| MbtH transformant | | Chrysogenin 13 | Chrysogenin 14 | Chrysogenin 15 | N-pyrovoylanthranilamid |
|---|---|---|---|---|---|
| COM | 1-2 | 1.17 | 1.00 | 1.02 | 3.22 |
| | 1-3 | 1.07 | 0.59 | 1.38 | 1.42 |
| | 1-5 | 1.35 | 0.98 | 1.32 | 2.14 |
| | 1-6 | 1.12 | 0.69 | 1.17 | 4.46 |
| COM | 2-2 | 1.40 | 0.68 | 1.40 | 5.12 |
| | 2-3 | 1.35 | 0.51 | 1.71 | 3.71 |
| | 2-4 | 0.96 | 0.75 | 1.20 | 1.25 |
| CDAI | 3-1 | 1.04 | 0.35 | 1.58 | 1.10 |
| | 3-2 | 1.31 | 0.95 | 1.39 | 1.03 |
| | 3-3 | 1.19 | 1.00 | 1.25 | 1.16 |
| | 3-4 | 1.21 | 0.62 | 1.49 | 0.82 |
| TCP13 | 4-1 | 1.72 | 1.13 | 1.05 | 1.67 |
| | 4-2 | 1.48 | 0.84 | 1.56 | 1.48 |
| | 4-3 | 1.42 | 0.97 | 1.37 | 1.83 |
| | 4-4 | 1.87 | 1.29 | 1.76 | 1.89 |
| | Tcp13_II(+) | 1.34 | 1.72 | 2.16 | 2.74 |
| | Tcp13_III(+) | 0.96 | 1.20 | 1.44 | 1.57 |
| TEG | 5-1 | 1.49 | 0.74 | 1.42 | 1.91 |
| | 5-2 | 1.45 | 0.74 | 1.67 | 1.69 |
| | 5-4 | 1.68 | 0.83 | 1.74 | 1.41 |
| | 5-5 | 1.41 | 0.82 | 1.40 | 1.70 |
| | Teg_II(+) | 1.32 | 1.45 | 0.74 | 1.43 |
| VEG8 | 6-3 | 1.52 | 0.83 | 1.62 | 1.53 |

| | | | | |
|---|---|---|---|---|
| All MbtH Avg. | 1.34 | 0.90 | 1.43 | 2.01 |

TABLE 5b

| MbtH transformant | | Chrysogenin | Chrysogenin B | Chrysogenin C | Chrysogenin 7 | Chrysogenin 11 | Chrysogenin 12 |
|---|---|---|---|---|---|---|---|
| COM | 1-2 | 2.36 | 2.50 | 2.08 | 1.83 | 1.53 | 2.12 |
| | 1-3 | 1.65 | 2.31 | 1.87 | 1.60 | 0.64 | 0.57 |
| | 1-5 | 2.18 | 2.31 | 2.02 | 1.76 | 0.43 | 0.16 |
| | 1-6 | 1.98 | 2.14 | 1.82 | 1.58 | 0.49 | 0.23 |
| | 2-2 | 2.63 | 3.40 | 2.42 | 2.17 | 0.05 | 0.00 |
| | 2-3 | 1.86 | 2.12 | 2.00 | 1.76 | 0.07 | 0.00 |
| | 2-4 | 1.56 | 2.19 | 1.56 | 1.38 | 1.03 | 1.06 |
| CDAI | 3-1 | 1.52 | 2.39 | 1.75 | 1.60 | 0.06 | 0.01 |
| | 3-2 | 1.73 | 1.88 | 1.74 | 1.54 | 1.00 | 1.36 |
| | 3-3 | 1.79 | 1.68 | 1.58 | 1.40 | 1.02 | 1.41 |
| | 3-4 | 1.41 | 1.88 | 1.61 | 1.42 | 0.41 | 0.19 |
| TCP13 | 4-1 | 0.86 | 0.81 | 2.84 | 2.52 | 1.90 | 1.96 |
| | 4-2 | 1.93 | 2.40 | 2.05 | 1.83 | 0.59 | 0.35 |
| | 4-3 | 2.11 | 2.14 | 1.95 | 1.79 | 1.18 | 1.40 |
| | 4-4 | 2.37 | 2.90 | 2.55 | 2.35 | 1.93 | 2.41 |
| | Tcp13_II(+) | 2.53 | 1.63 | 1.54 | 1.57 | 2.20 | 2.09 |
| | Tcp13_III(+) | 1.28 | 0.99 | 0.99 | 1.02 | 1.33 | 1.19 |
| TEG | 5-1 | 2.00 | 2.83 | 2.16 | 2.03 | 1.37 | 1.89 |
| | 5-2 | 2.16 | 2.47 | 1.93 | 1.81 | 0.13 | 0.02 |
| | 5-4 | 1.63 | 2.68 | 2.14 | 1.96 | 0.53 | 0.18 |
| | 5-5 | 1.76 | 2.05 | 1.77 | 1.65 | 0.44 | 0.26 |
| | Teg_II(+) | 3.03 | 2.25 | 1.65 | 1.66 | 3.59 | 3.96 |
| VEG8 | 6-3 | 1.81 | 2.43 | 1.94 | 1.84 | 1.48 | 1.52 |

| | | | | | | |
|---|---|---|---|---|---|---|
| All MbtH Avg. | 1.92 | 2.19 | 1.91 | 1.74 | 1.02 | 1.06 |

| MbtH transformant | | Chrysogenin 13 | Chrysogenin 14 | Chrysogenin 15 | N-pyrovoylanthranilamid |
|---|---|---|---|---|---|
| COM | 1-2 | 2.03 | 1.28 | 127.50 | 2.53 |
| | 1-3 | 1.83 | 0.50 | 161.55 | 1.39 |
| | 1-5 | 1.96 | 0.93 | 147.14 | 1.78 |
| | 1-6 | 1.78 | 0.77 | 143.17 | 2.01 |
| | 2-2 | 2.37 | 0.84 | 176.20 | 1.64 |
| | 2-3 | 1.98 | 0.86 | 162.11 | 1.47 |
| | 2-4 | 1.54 | 0.72 | 153.68 | 1.73 |
| CDAI | 3-1 | 1.72 | 0.20 | 170.28 | 1.19 |
| | 3-2 | 1.72 | 1.15 | 119.56 | 1.55 |
| | 3-3 | 1.54 | 1.41 | 55.93 | 1.62 |
| | 3-4 | 1.59 | 0.75 | 65.35 | 1.24 |
| TCP13 | 4-1 | 2.78 | 1.88 | 1.47 | 0.75 |
| | 4-2 | 2.02 | 0.79 | 127.09 | 1.86 |
| | 4-3 | 1.94 | 0.94 | 2.55 | 2.18 |
| | 4-4 | 2.51 | 1.77 | 74.40 | 1.68 |
| | Tcp13_II(+) | 1.52 | 1.71 | 1.80 | 2.41 |
| | Tcp13_III(+) | 0.99 | 0.98 | 1.11 | 1.29 |
| TEG | 5-1 | 2.13 | 0.84 | 119.48 | 2.95 |
| | 5-2 | 1.90 | 0.66 | 139.70 | 1.78 |
| | 5-4 | 2.11 | 1.06 | 136.62 | 1.93 |
| | 5-5 | 1.75 | 0.50 | 124.68 | 2.19 |
| | Teg_II(+) | 1.65 | 1.82 | 1.37 | 1.39 |
| VEG8 | 6-3 | 1.91 | 1.17 | 1.37 | 1.92 |

| | | | | |
|---|---|---|---|---|
| All MbtH Avg. | 1.88 | 1.02 | 96.27 | 1.76 |

TABLE 6a

| MbtH transformant | | HTD | DHTD | Roquefortine C | Roquefortine D | Roquefortine M | Roquefortine N |
|---|---|---|---|---|---|---|---|
| COM | 1-2 | 0.74 | 1.30 | 0.48 | 0.57 | 0.63 | 7.04 |
| | 1-3 | 0.29 | 0.66 | 0.26 | 0.17 | 0.32 | 4.87 |
| | 1-5 | 0.87 | 1.41 | 0.75 | 0.91 | 0.89 | 15.95 |
| | 1-6 | 0.44 | 0.80 | 0.40 | 0.43 | 0.30 | 19.36 |
| | 2-2 | 0.47 | 0.88 | 0.39 | 0.35 | 0.36 | 24.29 |
| | 2-3 | 0.66 | 1.07 | 0.51 | 0.36 | 0.60 | 6.58 |
| | 2-4 | 0.48 | 1.00 | 0.43 | 0.41 | 0.65 | 4.89 |
| CDAI | 3-1 | 0.47 | 0.84 | 0.47 | 0.34 | 0.40 | 4.86 |
| | 3-2 | 0.90 | 1.02 | 0.74 | 0.77 | 0.64 | 0.56 |
| | 3-3 | 0.84 | 1.24 | 0.83 | 0.71 | 0.63 | 2.16 |
| | 3-4 | 1.24 | 1.33 | 1.47 | 1.55 | 1.10 | 1.28 |
| TCP13 | 4-1 | 0.07 | 0.40 | 0.00 | 0.00 | 0.13 | 0.11 |
| | 4-2 | 0.73 | 0.95 | 0.59 | 0.59 | 0.58 | 0.84 |
| | 4-3 | 0.52 | 0.75 | 0.35 | 0.39 | 0.38 | 0.46 |
| | 4-4 | 0.51 | 1.12 | 0.83 | 0.95 | 0.71 | 0.59 |
| | Tcp13_II(+) | 1.93 | 7.51 | 1.73 | 2.07 | 2.70 | 4.59 |
| | Tcp13_III(+) | 1.60 | 5.71 | 1.19 | 1.42 | 6.27 | 3.12 |
| TEG | 5-1 | 0.23 | 0.61 | 0.37 | 0.38 | 0.31 | 0.89 |
| | 5-2 | 0.00 | 0.81 | 0.48 | 0.47 | 0.50 | 0.53 |
| | 5-4 | 0.00 | 0.93 | 0.63 | 0.73 | 0.64 | 0.67 |
| | 5-5 | 0.27 | 0.80 | 0.42 | 0.43 | 0.55 | 0.49 |
| | Teg_II(+) | 0.54 | 0.52 | 0.13 | 0.14 | 2.15 | 7.64 |
| VEG8 | 6-3 | 0.56 | 0.62 | 0.46 | 0.45 | 0.47 | 0.44 |

| | | | | | | |
|---|---|---|---|---|---|---|
| All MbtH Avg. | 0.62 | 1.40 | 0.60 | 0.63 | 0.95 | 4.88 |

| MbtH transformant | | Glandicoline A | Glandicoline B | Meleagrine | Neoxaline |
|---|---|---|---|---|---|
| COM | 1-2 | 0.47 | 0.66 | 0.64 | 0.10 |
| | 1-3 | 0.19 | 0.22 | 0.35 | 0.02 |
| | 1-5 | 1.00 | 0.94 | 1.03 | 0.26 |
| | 1-6 | 0.34 | 0.33 | 0.43 | 0.06 |
| | 2-2 | 0.14 | 0.35 | 0.40 | 0.07 |
| | 2-3 | 0.52 | 0.40 | 0.68 | 0.47 |
| | 2-4 | 0.47 | 0.76 | 0.52 | 0.16 |
| CDAI | 3-1 | 0.00 | 0.43 | 0.50 | 0.07 |
| | 3-2 | 0.89 | 0.86 | 0.84 | 0.23 |
| | 3-3 | 0.92 | 0.89 | 0.66 | 0.32 |
| | 3-4 | 1.72 | 2.32 | 1.90 | 0.58 |
| TCP13 | 4-1 | 0.00 | 0.07 | 0.08 | 0.15 |
| | 4-2 | 0.63 | 0.65 | 0.75 | 0.13 |
| | 4-3 | 0.44 | 0.52 | 0.51 | 0.10 |
| | 4-4 | 0.74 | 0.92 | 0.83 | 0.41 |
| | Tcp13_II(+) | 2.48 | 2.26 | 2.71 | 0.00 |
| | Tcp13_III(+) | 1.82 | 1.65 | 1.98 | 0.00 |
| TEG | 5-1 | 0.15 | 0.71 | 0.55 | 0.08 |
| | 5-2 | 0.38 | 0.54 | 0.57 | 0.16 |
| | 5-4 | 0.78 | 1.13 | 1.06 | 0.17 |
| | 5-5 | 0.39 | 0.58 | 0.69 | 0.20 |
| | Teg_II(+) | 0.14 | 0.18 | 0.46 | 0.00 |
| VEG8 | 6-3 | 0.53 | 0.45 | 0.47 | 0.22 |

| | | | | |
|---|---|---|---|---|
| All MbtH Avg. | 0.66 | 0.77 | 0.81 | 0.17 |

TABLE 6b

| MbtH transformant | | HTD | DHTD | Roquefortine C | Roquefortine D | Roquefortine M | Roquefortine N |
|---|---|---|---|---|---|---|---|
| COM | 1-2 | 1.06 | 1.16 | 0.95 | 1.06 | 0.99 | 0.93 |
| | 1-3 | 0.93 | 13.09 | 6.92 | 0.37 | 2.96 | 0.90 |
| | 1-5 | 2.42 | 5.29 | 7.09 | 1.68 | 2.92 | 1.56 |
| | 1-6 | 0.98 | 1.02 | 1.73 | 0.77 | 0.85 | 0.64 |
| | 2-2 | 1.19 | 2.60 | 3.13 | 0.63 | 1.09 | 0.83 |
| | 2-3 | 0.90 | 1.38 | 1.27 | 0.41 | 1.52 | 0.67 |
| | 2-4 | 3.30 | 40.35 | 16.99 | 2.11 | 13.48 | 4.67 |
| CDAI | 3-1 | 0.97 | 1.98 | 2.00 | 0.59 | 1.10 | 0.72 |
| | 3-2 | 1.59 | 1.74 | 1.34 | 1.07 | 1.46 | 1.02 |
| | 3-3 | 1.54 | 1.62 | 1.63 | 1.14 | 1.59 | 1.14 |
| | 3-4 | 2.43 | 4.27 | 4.18 | 2.11 | 3.46 | 2.26 |
| TCP13 | 4-1 | 0.38 | 7.81 | 1.89 | 0.27 | 3.08 | 0.45 |
| | 4-2 | 1.09 | 1.73 | 1.17 | 0.70 | 1.25 | 0.89 |
| | 4-3 | 2.16 | 5.64 | 6.16 | 1.59 | 3.13 | 1.32 |
| | 4-4 | 1.04 | 1.45 | 0.51 | 0.82 | 1.11 | 1.02 |
| | Tcp13_II(+) | 1.51 | 2.56 | 0.00 | 1.71 | 5.57 | 2.33 |
| | Tcp13_III(+) | 1.12 | 1.52 | 0.00 | 0.90 | 3.04 | 1.19 |
| TEG | 5-1 | 2.10 | 5.43 | 4.52 | 1.54 | 2.56 | 1.72 |
| | 5-2 | 1.68 | 3.93 | 5.93 | 1.13 | 1.61 | 0.96 |
| | 5-4 | 0.96 | 3.01 | 1.33 | 0.77 | 1.47 | 1.40 |
| | 5-5 | 2.52 | 12.46 | 18.94 | 1.56 | 3.85 | 1.49 |
| | Teg_II(+) | 1.68 | 0.96 | 0.00 | 3.18 | 2.79 | 1.79 |
| VEG8 | 6-3 | 1.34 | 2.19 | 2.57 | 1.05 | 1.50 | 0.81 |

| | | | | | | |
|---|---|---|---|---|---|---|
| All MbtH Avg. | 1.52 | 5.36 | 3.92 | 1.18 | 2.71 | 1.34 |

| MbtH transformant | | Glandicoline A | Glandicoline B | Meleagrine | Neoxaline |
|---|---|---|---|---|---|
| COM | 1-2 | 0.68 | 1.13 | 1.13 | 0.91 |
| | 1-3 | 1.92 | 4.02 | 4.53 | 3.04 |
| | 1-5 | 2.10 | 2.75 | 3.06 | 3.87 |
| | 1-6 | 0.68 | 0.92 | 1.04 | 0.39 |
| | 2-2 | 0.78 | 1.27 | 1.20 | 0.27 |
| | 2-3 | 0.85 | 0.77 | 0.74 | 0.56 |
| | 2-4 | 8.87 | 27.76 | 25.61 | 8.75 |
| CDAI | 3-1 | 0.39 | 1.15 | 1.09 | 0.21 |
| | 3-2 | 1.29 | 1.03 | 1.00 | 0.91 |
| | 3-3 | 1.30 | 1.00 | 1.00 | 2.30 |
| | 3-4 | 2.66 | 3.33 | 3.07 | 3.56 |
| TCP13 | 4-1 | 0.61 | 0.55 | 1.25 | 2.31 |
| | 4-2 | 0.75 | 0.95 | 0.91 | 0.52 |
| | 4-3 | 2.33 | 2.89 | 3.69 | 3.65 |
| | 4-4 | 1.00 | 0.63 | 0.45 | 0.48 |
| | Tcp13_II(+) | 2.08 | 1.76 | 1.69 | 0.00 |
| | Tcp13_III(+) | 1.25 | 3.30 | 1.73 | 0.00 |
| TEG | 5-1 | 1.41 | 3.66 | 3.91 | 1.20 |
| | 5-2 | 1.15 | 1.84 | 2.17 | 0.88 |
| | 5-4 | 0.94 | 1.60 | 1.39 | 0.75 |
| | 5-5 | 3.42 | 9.56 | 11.42 | 6.74 |
| | Teg_II(+) | 1.61 | 1.40 | 1.33 | 0.00 |
| VEG8 | 6-3 | 1.18 | 1.22 | 1.59 | 1.26 |

| | | | | |
|---|---|---|---|---|
| All MbtH Avg. | 1.71 | 3.24 | 3.26 | 1.85 |

TABLE 7

| | | Day 2 | | | Day 5 | | |
|---|---|---|---|---|---|---|---|
| MbtH transformant | | 6-Aminopenicillanic acid | LLD-ACV | Penicillin G | 6-Aminopenicillanic acid | LLD-ACV | Penicillin G |
| COM | 7-3 | 1.02 | 0.28 | 1.40 | 2.87 | 22.70 | 1.76 |
| | Com_IV(+) | 1.80 | 2.15 | 1.76 | 1.14 | 0.99 | 1.27 |
| | Com_XIV(+) | 2.06 | 3.57 | 1.56 | 1.06 | 0.93 | 1.02 |
| | Com_XII(+) | 1.31 | 1.26 | 1.12 | 1.07 | 1.52 | 1.15 |
| | 8-1 | 1.16 | 0.95 | 1.37 | 1.36 | 4.07 | 2.24 |
| | 8-4 | 1.66 | 0.99 | 1.15 | 1.62 | 1.47 | 1.87 |
| | Com_VIII(+) | 2.05 | 4.58 | 1.57 | 1.01 | 0.97 | 1.11 |
| | Com_II(+) | 2.76 | 5.23 | 1.92 | 1.51 | 1.38 | 1.45 |
| | Com_XI(+) | 1.89 | 3.05 | 1.60 | 1.06 | 1.05 | 1.12 |
| CDAI | 9-2 | 2.05 | 0.95 | 0.89 | 1.51 | 3.88 | 2.52 |
| | 9-3 | 2.08 | 1.47 | 0.99 | 1.29 | 2.78 | 1.62 |
| | 9-4 | 1.78 | 1.08 | 1.02 | 1.33 | 5.39 | 2.46 |
| | 9-5 | 0.91 | 0.50 | 2.20 | 1.85 | 12.47 | 3.29 |
| TCP13 | 10-2 | 1.37 | 1.28 | 1.25 | 1.48 | 16.08 | 0.79 |
| | 10-5 | 1.53 | 1.27 | 1.13 | 0.99 | 10.53 | 1.38 |
| | 10-6 | 1.25 | 1.40 | 0.88 | 0.76 | 2.88 | 2.08 |
| TEG | 11-2 | 1.69 | 1.31 | 0.74 | 1.23 | 1.05 | 0.94 |
| | 11-3 | 1.16 | 1.04 | 0.80 | 0.92 | 2.64 | 1.79 |
| | 11-5 | 1.75 | 0.90 | 0.48 | 0.73 | 10.86 | 1.67 |
| VEG8 | 12-4 | 1.39 | 0.81 | 0.35 | 0.98 | 2.32 | 1.80 |
| | Veg8_XI(+) | 1.42 | 1.76 | 1.51 | 0.98 | 1.19 | 1.09 |

| | | | | | | |
|---|---|---|---|---|---|---|
| All MbtH Avg. | 1.62 | 1.71 | 1.22 | 1.27 | 5.10 | 1.64 |

TABLE 8a

| MbtH transformant | | Chrysogenin | Chrysogenin B | Chrysogenin C | Chrysogenin 7 | Chrysogenin 11 | Chrysogenin 12 |
|---|---|---|---|---|---|---|---|
| COM | 7-3 | 1.83 | 0.79 | 0.38 | 0.35 | 0.45 | 0.20 |
| | Com_IV(+) | 0.38 | 0.46 | 0.64 | 0.65 | 0.49 | 0.41 |
| | Com_XIV(+) | 0.69 | 1.25 | 1.59 | 1.60 | 1.08 | 1.11 |
| | Com_XII(+) | 0.41 | 0.65 | 1.09 | 1.10 | 0.80 | 0.67 |
| | 8-1 | 0.92 | 1.45 | 1.14 | 1.11 | 1.02 | 0.95 |
| | 8-4 | 1.22 | 1.46 | 1.32 | 1.29 | 1.44 | 1.18 |
| | Com_VIII(+) | 0.44 | 0.95 | 1.07 | 1.07 | 0.77 | 0.71 |
| | Com_II(+) | 0.31 | 0.44 | 0.79 | 0.79 | 0.60 | 0.42 |
| | Com_XI(+) | 0.30 | 0.43 | 0.62 | 0.62 | 0.44 | 0.42 |
| CDAI | 9-2 | 1.06 | 1.44 | 1.34 | 1.27 | 1.33 | 1.12 |
| | 9-3 | 1.09 | 1.30 | 1.26 | 1.22 | 1.33 | 1.00 |
| | 9-4 | 1.21 | 1.27 | 1.22 | 1.19 | 1.32 | 0.97 |
| | 9-5 | 1.59 | 0.82 | 0.61 | 0.59 | 0.12 | 0.11 |
| TCP13 | 10-2 | 1.07 | 1.32 | 1.09 | 1.09 | 0.84 | 0.77 |
| | 10-5 | 1.00 | 1.12 | 1.06 | 1.07 | 0.94 | 0.71 |
| | 10-6 | 0.87 | 1.49 | 1.15 | 1.14 | 1.01 | 0.98 |
| TEG | 11-2 | 0.98 | 1.11 | 1.13 | 1.13 | 1.10 | 0.99 |
| | 11-3 | 1.37 | 1.40 | 1.17 | 1.15 | 1.22 | 1.08 |
| | 11-5 | 0.61 | 0.64 | 0.88 | 0.87 | 0.71 | 0.40 |
| VEG8 | 12-4 | 0.98 | 1.05 | 0.99 | 0.99 | 1.15 | 0.81 |
| | Veg8_XI(+) | 0.16 | 0.19 | 0.25 | 0.25 | 0.15 | 0.17 |

| | | | | | | |
|---|---|---|---|---|---|---|
| All MbtH Avg. | 0.88 | 1.00 | 0.99 | 0.98 | 0.87 | 0.72 |

| MbtH transformant | | Chrysogenin 13 | Chrysogenin 14 | Chrysogenin 15 | N-pyrovoylanthranilamid |
|---|---|---|---|---|---|
| COM | 7-3 | 0.37 | 1.73 | 0.86 | 1.39 |
| | Com_IV(+) | 0.63 | 0.59 | 3.79 | 0.89 |
| | Com_XIV(+) | 1.57 | 0.57 | 3.45 | 0.85 |
| | Com_XII(+) | 1.09 | 0.75 | 3.53 | 0.59 |
| | 8-1 | 1.13 | 1.00 | 1.18 | 0.78 |
| | 8-4 | 1.31 | 1.35 | 0.62 | 1.42 |
| | Com_VIII(+) | 1.06 | 0.44 | 6.02 | 0.48 |
| | Com_II(+) | 0.78 | 0.50 | 6.07 | 0.45 |
| | Com_XI(+) | 0.61 | 0.57 | 4.25 | 0.58 |
| CDAI | 9-2 | 1.32 | 1.25 | 1.21 | 1.28 |
| | 9-3 | 1.25 | 1.27 | 0.55 | 1.07 |
| | 9-4 | 1.23 | 1.35 | 0.59 | 1.11 |
| | 9-5 | 0.60 | 1.96 | 0.46 | 1.50 |
| TCP13 | 10-2 | 1.09 | 1.29 | 1.05 | 0.90 |
| | 10-5 | 1.05 | 1.06 | 1.13 | 0.88 |
| | 10-6 | 1.14 | 1.26 | 0.91 | 0.88 |
| TEG | 11-2 | 1.14 | 0.92 | 1.14 | 1.09 |
| | 11-3 | 1.17 | 1.77 | 0.98 | 1.30 |
| | 11-5 | 0.87 | 1.23 | 1.00 | 0.80 |
| VEG8 | 12-4 | 0.99 | 0.94 | 0.93 | 1.00 |
| | Veg8_XI(+) | 0.23 | 0.36 | 3.77 | 0.37 |

| | | | | |
|---|---|---|---|---|
| All MbtH Avg. | 0.98 | 1.06 | 2.07 | 0.93 |

TABLE 8b

| MbtH transformant | | Chrysogenin | Chrysogenin B | Chrysogenin C | Chrysogenin 7 | Chrysogenin 11 | Chrysogenin 12 |
|---|---|---|---|---|---|---|---|
| COM | 7-3 | 2.24 | 1.67 | 1.25 | 1.19 | 139.28 | 316.81 |
| | Com_IV(+) | 0.32 | 0.35 | 0.42 | 0.44 | 0.34 | 0.28 |
| | Com_XIV(+) | 0.30 | 0.42 | 0.56 | 0.57 | 0.41 | 0.35 |
| | Com_XII(+) | 0.67 | 1.04 | 1.23 | 1.28 | 0.72 | 0.79 |
| | 8-1 | 1.12 | 1.75 | 1.26 | 1.17 | 25.32 | 252.26 |
| | 8-4 | 1.27 | 1.67 | 1.45 | 1.36 | 9.40 | 30.71 |
| | Com_VIII(+) | 0.41 | 0.72 | 0.67 | 0.69 | 0.45 | 0.44 |
| | Com_II(+) | 0.27 | 0.35 | 0.50 | 0.51 | 0.36 | 0.29 |
| | Com_XI(+) | 0.27 | 0.32 | 0.38 | 0.39 | 0.26 | 0.28 |
| CDAI | 9-2 | 1.16 | 1.60 | 1.49 | 1.42 | 31.64 | 266.13 |
| | 9-3 | 1.25 | 1.45 | 1.35 | 1.28 | 50.70 | 361.63 |
| | 9-4 | 1.37 | 1.49 | 1.46 | 1.39 | 46.45 | 413.07 |
| | 9-5 | 2.00 | 1.24 | 1.00 | 1.00 | 0.72 | 9.12 |
| TCP13 | 10-2 | 1.21 | 1.61 | 1.29 | 1.25 | 4.79 | 157.71 |
| | 10-5 | 0.97 | 1.23 | 1.10 | 1.05 | 55.13 | 464.86 |
| | 10-6 | 0.94 | 1.67 | 1.36 | 1.33 | 3.51 | 36.70 |
| TEG | 11-2 | 0.95 | 1.05 | 1.05 | 1.03 | 1.37 | 2.59 |
| | 11-3 | 1.77 | 1.65 | 1.40 | 1.41 | 13.08 | 46.13 |
| | 11-5 | 0.55 | 0.65 | 0.81 | 0.80 | 32.50 | 265.89 |
| VEG8 | 12-4 | 1.04 | 1.07 | 0.98 | 1.00 | 28.96 | 189.14 |
| | Veg8_XI(+) | 0.12 | 0.13 | 0.15 | 0.15 | 0.10 | 0.11 |

| | | | | | | |
|---|---|---|---|---|---|---|
| All MbtH Avg. | 0.96 | 1.10 | 1.01 | 0.99 | 21.21 | 134.06 |

| MbtH transformant | | Chrysogenin 13 | Chrysogenin 14 | Chrysogenin 15 | N-pyrovoylanthranilamid |
|---|---|---|---|---|---|
| COM | 7-3 | 1.24 | 4.41 | 65.38 | 2.20 |
| | Com_IV(+) | 0.42 | 0.45 | 1.66 | 0.63 |
| | Com_XIV(+) | 0.56 | 0.50 | 1.20 | 0.41 |
| | Com_XII(+) | 1.22 | 0.56 | 1.31 | 0.76 |
| | 8-1 | 1.26 | 0.63 | 87.07 | 1.59 |
| | 8-4 | 1.44 | 1.27 | 78.07 | 2.21 |
| | Com_VIII(+) | 0.68 | 0.43 | 1.27 | 0.51 |
| | Com_II(+) | 0.49 | 0.47 | 1.80 | 0.44 |
| | Com_XI(+) | 0.37 | 0.48 | 1.76 | 0.51 |
| CDAI | 9-2 | 1.47 | 2.81 | 66.77 | 1.82 |
| | 9-3 | 1.33 | 0.90 | 126.30 | 2.07 |
| | 9-4 | 1.44 | 2.83 | 134.44 | 1.85 |
| | 9-5 | 1.00 | 5.99 | 116.24 | 1.69 |
| TCP13 | 10-2 | 1.30 | 1.00 | 68.05 | 1.79 |
| | 10-5 | 1.09 | 0.75 | 141.61 | 1.53 |
| | 10-6 | 1.36 | 2.48 | 92.90 | 1.09 |
| TEG | 11-2 | 1.04 | 0.89 | 64.09 | 0.97 |
| | 11-3 | 1.41 | 2.86 | 116.07 | 1.78 |
| | 11-5 | 0.81 | 1.16 | 0.64 | 0.85 |
| VEG8 | 12-4 | 0.98 | 1.09 | 103.18 | 1.52 |
| | Veg8_XI(+) | 0.14 | 0.26 | 1.33 | 0.24 |

| | | | | |
|---|---|---|---|---|
| All MbtH Avg. | 1.00 | 1.53 | 60.53 | 1.26 |

TABLE 9a

| MbtH transformant | | HTD | DHTD | Roquefortine C | Roquefortine D | Roquefortine M | Roquefortine N |
|---|---|---|---|---|---|---|---|
| COM | 7-3 | 2.31 | 11.33 | 4.84 | 3.07 | 10.40 | 7.90 |
| | Com_IV(+) | 1.72 | 0.75 | 0.42 | 1.42 | 1.08 | 1.04 |
| | Com_XIV(+) | 1.19 | 0.95 | 0.40 | 0.88 | 0.94 | 0.96 |
| | Com_XII(+) | 1.74 | 0.78 | 0.49 | 1.56 | 1.13 | 1.22 |
| | 8-1 | 1.53 | 1.67 | 3.04 | 3.53 | 1.45 | 2.34 |
| | 8-4 | 2.32 | 1.95 | 3.02 | 4.74 | 2.10 | 2.22 |
| | Com_VIII(+) | 1.24 | 0.91 | 0.48 | 0.75 | 0.91 | 1.03 |
| | Com_II(+) | 1.62 | 0.82 | 0.65 | 1.25 | 1.23 | 1.30 |
| | Com_XI(+) | 1.53 | 1.07 | 0.62 | 1.16 | 1.30 | 1.36 |
| CDAI | 9-2 | 3.08 | 2.38 | 4.15 | 8.03 | 2.83 | 4.38 |
| | 9-3 | 2.89 | 2.93 | 4.20 | 5.83 | 3.06 | 4.41 |
| | 9-4 | 3.40 | 2.31 | 4.62 | 8.95 | 3.50 | 4.02 |
| | 9-5 | 4.48 | 9.10 | 9.52 | 9.75 | 12.29 | 9.00 |
| TCP13 | 10-2 | 2.64 | 2.22 | 3.13 | 5.70 | 2.72 | 2.94 |
| | 10-5 | 2.32 | 2.35 | 4.70 | 5.46 | 2.64 | 2.89 |
| | 10-6 | 1.53 | 1.76 | 1.66 | 2.20 | 2.15 | 1.66 |
| TEG | 11-2 | 2.35 | 2.22 | 3.80 | 3.90 | 2.20 | 1.56 |
| | 11-3 | 2.33 | 1.77 | 2.74 | 4.90 | 2.39 | 2.16 |
| | 11-5 | 9.35 | 8.24 | 35.42 | 46.90 | 12.51 | 13.61 |
| VEG8 | 12-4 | 2.90 | 2.66 | 5.89 | 9.46 | 3.40 | 4.65 |
| | Veg8_XI(+) | 2.08 | 1.62 | 1.20 | 2.14 | 2.44 | 2.59 |

| | | | | | | |
|---|---|---|---|---|---|---|
| All MbtH Avg. | 2.60 | 2.85 | 4.52 | 6.26 | 3.46 | 3.49 |

| MbtH transformant | | Glandicoline A | Glandicoline B | Meleagrine | Neoxaline |
|---|---|---|---|---|---|
| COM | 7-3 | 38.28 | 6.14 | 5.92 | 21.95 |
| | Com_IV(+) | 0.78 | 0.54 | 0.90 | 0.89 |
| | Com_XIV(+) | 0.47 | 0.63 | 0.64 | 0.63 |
| | Com_XII(+) | 0.54 | 0.74 | 0.98 | 0.92 |
| | 8-1 | 12.49 | 3.52 | 2.77 | 3.58 |
| | 8-4 | 14.12 | 4.22 | 3.59 | 4.29 |
| | Com_VIII(+) | 0.28 | 0.70 | 0.56 | 0.48 |
| | Com_II(+) | 0.40 | 0.96 | 0.84 | 0.78 |
| | Com_XI(+) | 0.64 | 1.03 | 1.02 | 1.00 |
| CDAI | 9-2 | 24.61 | 7.69 | 5.20 | 7.13 |
| | 9-3 | 13.70 | 4.12 | 3.64 | 4.80 |
| | 9-4 | 21.46 | 5.89 | 4.80 | 6.20 |
| | 9-5 | 43.46 | 8.28 | 5.93 | 11.55 |
| TCP13 | 10-2 | 17.13 | 4.44 | 3.29 | 4.49 |
| | 10-5 | 14.31 | 5.08 | 3.96 | 4.81 |
| | 10-6 | 6.58 | 1.97 | 2.17 | 3.87 |
| TEG | 11-2 | 8.91 | 3.42 | 2.96 | 3.88 |
| | 11-3 | 12.21 | 3.28 | 2.31 | 3.04 |
| | 11-5 | 88.86 | 48.18 | 35.01 | 52.04 |
| VEG8 | 12-4 | 20.75 | 7.93 | 6.16 | 8.14 |
| | Veg8_XI(+) | 1.48 | 1.84 | 2.05 | 2.12 |

| | | | | |
|---|---|---|---|---|
| All MbtH Avg. | 16.26 | 5.74 | 4.51 | 6.98 |

TABLE 9b

| MbtH transformant | | HTD | DHTD | Roquefortine C | Roquefortine D | Roquefortine M | Roquefortine N |
|---|---|---|---|---|---|---|---|
| COM | 7-3 | 2.20 | 2.77 | 0.56 | 7.68 | 11.07 | 16.72 |
| | Com_IV(+) | 0.96 | 1.20 | 0.54 | 0.73 | 0.72 | 0.91 |
| | Com_XIV(+) | 0.74 | 0.80 | 0.31 | 0.56 | 0.38 | 0.55 |
| | Com_XII(+) | 0.57 | 0.69 | 0.30 | 0.38 | 0.33 | 0.41 |
| | 8-1 | 1.53 | 6.55 | 5.34 | 3.38 | 6.78 | 4.98 |
| | 8-4 | 2.44 | 2.78 | 3.15 | 5.77 | 3.54 | 3.57 |
| | Com_VIII(+) | 0.62 | 0.68 | 0.39 | 0.24 | 0.06 | 0.25 |
| | Com_II(+) | 0.88 | 1.13 | 0.65 | 0.51 | 0.38 | 0.57 |
| | Com_XI(+) | 0.80 | 0.86 | 0.31 | 0.51 | 0.33 | 0.79 |
| CDAI | 9-2 | 3.69 | 3.00 | 1.88 | 10.05 | 4.00 | 5.90 |
| | 9-3 | 1.79 | 2.42 | 3.10 | 4.10 | 3.13 | 3.76 |
| | 9-4 | 2.43 | 1.40 | 0.73 | 6.38 | 2.56 | 4.36 |
| | 9-5 | 3.39 | 3.11 | 1.00 | 9.31 | 5.14 | 13.18 |
| TCP13 | 10-2 | 1.39 | 1.55 | 1.35 | 3.58 | 2.69 | 3.81 |
| | 10-5 | 2.03 | 5.44 | 4.91 | 5.29 | 6.45 | 4.48 |
| | 10-6 | 0.43 | 0.38 | 0.07 | 0.65 | 0.68 | 1.58 |
| TEG | 11-2 | 1.36 | 1.52 | 1.24 | 2.24 | 2.31 | 2.71 |
| | 11-3 | 0.86 | 0.56 | 0.20 | 1.90 | 1.13 | 2.53 |
| | 11-5 | 5.74 | 9.00 | 2.50 | 30.42 | 19.56 | 29.62 |
| VEG8 | 12-4 | 2.81 | 3.75 | 1.73 | 8.22 | 5.54 | 6.43 |
| | Veg8_XI(+) | 1.17 | 2.02 | 1.31 | 1.09 | 1.58 | 1.61 |

| | | | | | | |
|---|---|---|---|---|---|---|
| All MbtH Avg. | 1.80 | 2.46 | 1.50 | 4.90 | 3.73 | 5.18 |

| MbtH transformant | | Glandicoline A | Glandicoline B | Meleagrine | Neoxaline |
|---|---|---|---|---|---|
| COM | 7-3 | 7.58 | 3.12 | 1.85 | 4.55 |
| | Com_IV(+) | 0.69 | 0.90 | 0.92 | 0.94 |
| | Com_XIV(+) | 0.51 | 0.40 | 0.51 | 0.46 |
| | Com_XII(+) | 0.33 | 0.29 | 0.35 | 0.34 |
| | 8-1 | 7.83 | 8.38 | 8.01 | 9.37 |
| | 8-4 | 3.82 | 4.02 | 4.16 | 4.66 |
| | Com_VIII(+) | 0.21 | 0.13 | 0.16 | 0.15 |
| | Com_II(+) | 0.54 | 0.39 | 0.44 | 0.45 |
| | Com_XI(+) | 0.41 | 0.51 | 0.57 | 0.57 |
| CDAI | 9-2 | 5.07 | 4.06 | 3.84 | 4.24 |
| | 9-3 | 4.04 | 3.63 | 3.71 | 4.13 |
| | 9-4 | 2.91 | 1.95 | 1.69 | 1.79 |
| | 9-5 | 7.80 | 2.37 | 1.28 | 2.51 |
| TCP13 | 10-2 | 2.76 | 2.15 | 2.17 | 3.26 |
| | 10-5 | 8.25 | 9.02 | 8.23 | 9.25 |
| | 10-6 | 0.51 | 0.27 | 0.14 | 0.26 |
| TEG | 11-2 | 1.92 | 1.85 | 1.71 | 1.83 |
| | 11-3 | 0.91 | 0.53 | 0.32 | 0.36 |
| | 11-5 | 18.48 | 20.41 | 13.58 | 32.20 |
| VEG8 | 12-4 | 7.62 | 6.92 | 5.77 | 6.82 |
| | Veg8_XI(+) | 1.48 | 1.77 | 1.76 | 1.84 |

| | | | | |
|---|---|---|---|---|
| All MbtH Avg. | 3.98 | 3.48 | 2.91 | 4.28 |

1. A method to improve the production of a secondary metabolite or a precursor occurring in the pathway leading to said secondary metabolite catalyzed by a non-ribosomal peptide synthetase comprising contacting in a eukaryotic host said non-ribosomal peptide synthetase with an MbtH-like protein, characterized in that said non-ribosomal peptide synthetase is from eukaryotic origin and is not a hybrid.

2. The method according to claim 1 wherein said eukaryotic host is a fungus.

3. The method according to claim 1 wherein said secondary metabolite is a β-lactam, a pigment or a mycotoxin.

4. The method according to claim 3 wherein said β-lactam is 6-aminopenicillanic acid, 7-aminodesacetoxycephalosporanic acid, adipyl 7-aminodesacetoxycephalosporanic acid, cephalosporin C, penicillin G or penicillin V, wherein said pigment is a chrysogenin or wherein said mycotoxin is a roquefortin.

5. The method according to claim 2 wherein said fungus is Penicillium chrysogenum.

6. The method according to claim 5 wherein said eukaryotic host is a multi copy strain.

7. The method according to claim 1 wherein said MbtH-like protein has SEQ ID NO: 1-7 or a sequence that is at least 70% homologous to SEQ ID NO: 1-7.

8. The method according to claim 1 wherein said MbtH-like protein comprises the amino acid code of SEQ ID NO: 17.

9. The method according to claim 8 wherein said MbtH-like protein comprises the amino acid code of any one of SEQ ID NO: 18 to SEQ ID NO: 53 or SEQ ID NO: 57 to SEQ ID NO: 92.

10. A composition comprising a eukaryotic non-ribosomal peptide synthetase that is not a hybrid and a prokaryotic MbtH.

11. The composition according to claim 10 which is
SEQ ID NO: 54 with SEQ ID NO: 1; or
SEQ ID NO: 54 with SEQ ID NO: 3; or
SEQ ID NO: 54 with SEQ ID NO: 4; or
SEQ ID NO: 54 with SEQ ID NO: 6; or
SEQ ID NO: 54 with SEQ ID NO: 7; or
SEQ ID NO: 55 with SEQ ID NO: 1; or
SEQ ID NO: 55 with SEQ ID NO: 3; or
SEQ ID NO: 55 with SEQ ID NO: 4; or
SEQ ID NO: 55 with SEQ ID NO: 6; or
SEQ ID NO: 55 with SEQ ID NO: 7; or
SEQ ID NO: 56 with SEQ ID NO: 1; or
SEQ ID NO: 56 with SEQ ID NO: 3; or
SEQ ID NO: 56 with SEQ ID NO: 4; or
SEQ ID NO: 56 with SEQ ID NO: 6; or
SEQ ID NO: 56 with SEQ ID NO: 7; or
sequences that are at last 90% homologous thereto.

12. A eukaryotic host cell comprising a non-ribosomal peptide synthetase that is not a hybrid and a polynucleotide allowing the expression of an MbtH-like protein, characterized in that said non-ribosomal peptide synthetase is from eukaryotic origin.

13. The eukaryotic host cell according to claim 12 wherein said MbtH-like protein has SEQ ID NO: 1-7 or a sequence that is at least 70% homologous to SEQ ID NO: 1-7.

14. The eukaryotic host cell according to claim 12 wherein said MbtH-like protein comprises the amino acid code of SEQ ID NO: 17.

15. The eukaryotic host cell according to claim 12 which is Penicillium chrysogenum.
